(19) United States
(12) Patent Application Publication
(10) Pub. No.: US 2025/0034627 A1
(43) Pub. Date: Jan. 30, 2025

(54) METHODS FOR AMPLIFICATION-FREE ENRICHMENT

(71) Applicant: GUARDANT HEALTH, INC., Palo Alto, CA (US)

(72) Inventors: Anne M. DEMAREE, Palo Alto, CA (US); Andrew KENNEDY, San Diego, CA (US)

(21) Appl. No.: 18/783,866

(22) Filed: Jul. 25, 2024

Related U.S. Application Data
(60) Provisional application No. 63/515,591, filed on Jul. 26, 2023.

(51) Int. Cl. C12Q 1/683; C12Q 1/6874

(52) U.S. Cl. CPC C12Q 1/683; C12Q 1/6874

(57) ABSTRACT

An analysis technique for enriching one or more target regions in molecules of deoxyribonucleic acid (DNA) in a sample (such as a tissue sample or cell-free DNA) is described. Notably, in this analysis technique, adapters are attached to ends of the molecules of DNA. Then, the molecules of DNA are selectively cut in one or more off-target regions in the molecules of DNA. The selective cutting may use a Cas9 protein or a restriction enzyme. Moreover, the one or more off-target regions in the molecules of DNA are depleted or removed, where the off-target regions are different from the target regions. Next, after the depleting or removing operation, motor adapters are attached to a remainder of the molecules of DNA. In some embodiments, the one or more target regions are analyzed by pulling or extracting the remainder of the molecules of DNA through an analysis membrane using the motor adapters.

### CROSS-REFERENCE

This application claims the benefit of U.S. Provisional Application No. 63/515,591, filed on Jul. 26, 2023, the contents of which are incorporated herein by reference in its entirety.

### FIELD

The described embodiments relate to techniques for processing a sample. Notably, certain embodiments relate to techniques for processing a sample by selectively cutting and depletion or removal in one or more off-target regions in molecules of deoxyribonucleic acid (DNA), attaching motor adapters to locations on the remainder of the molecules of DNA, and then analyzing the one or more target regions by pulling out or extracting the remainder of the molecules of DNA through an analysis membrane using the motor adapters.

### BACKGROUND

Advances in genetic analysis are enabling improved diagnosis and treatment of diseases. Notably, the analysis of genetic markers (such as the patterns or sequences of nucleotides or the genotype) in DNA from a sample can improve the detection of diseases (such as cancer), as well as determine classifications that allow personalized or individual-specific treatments (which is sometimes referred to as ‘precision medicine’).

However, accurate analysis of DNA is often complicated. For example, it can be difficult to accurately analyze fragments of DNA or cell-free DNA having short lengths (such as less than 200 bases). Typically, fragments of DNA or cell-free DNA are analyzed by amplification.

But it can be difficult to target the amplification to areas of interest on molecules of DNA, which can degrade the accuracy of the analysis. Incorrect results undermine confidence in DNA results, and can result in unnecessary or untimely therapeutic interventions, patient suffering and increased patient mortality.

### SUMMARY

A method for processing a sample is described. In this method, molecules of nucleic acids, such as DNA, in the sample are received or collected. Then, adapters are attached to ends of the molecules of DNA. Moreover, the molecules of DNA are selectively cut at one or more off-target regions, resulting in fragment ends. Next, the off-target regions are removed, increasing a fraction of or leaving only the target regions of the molecules of DNA that contain methylation or binding states. Moreover, following the depletion or removal operation, motor adapters are attached to locations on a remainder of the molecules of DNA. Furthermore, the one or more target regions are analyzed by a nucleic acid sequencer. In some embodiments, the one or more target regions are sequenced using an analysis membrane.

Note that the adapters may prevent the selective cutting. For example, the adapters may include ligation adapters, and a given off-target region may be separated from a given target region by a given ligation adapter. In some embodiments, the selective cutting uses a Cas9 protein or a restriction enzyme.

Moreover, the depletion or removal may include: digesting the off-target regions in the molecules of DNA; or pulling or extracting out of solution the digested off-target regions in the molecules of DNA. For example, the digestion may include exonuclease digestion. Alternatively or additionally, the pulling or extracting out may use magnetic beads, such as streptavidin magnetic beads.

Furthermore, the off-target regions may include at least a subset of the fragment ends in the molecules of DNA.

Additionally, the analysis membrane may include a nanopore membrane.

In some embodiments, the molecules of DNA may include cell-free DNA having lengths less than 200 bases.

Moreover, the method may exclude amplification of the one or more target regions.

Furthermore, the analysis may include determining the molecules of DNA in the one or more target regions. For example, the analysis may include determining the methylation states or the binding states of the one or more target regions.

Another embodiment provides a computer system (with one or more computers) that performs or executes instructions for at least some of the operations in the method.

Another embodiment provides a computer-readable storage medium for use with the computer system. When executed by the computer system, this computer-readable storage medium causes the computer system to perform or execute instructions for at least some of the aforementioned operations in the method.

In certain embodiments, systems biology approaches directed to the study of complex diseases can integrate genomic and/or epigenomic information with proteomics (proteins and their post-translational modifications) or transcriptomic data leading to new biological and insights of disease initiation, progression, malignant transformation, and therapeutic outcomes.

In some embodiments, the results of the systems and methods disclosed herein are used as an input to generate a report. The report may be in a paper or electronic format. For example, genetic results as determined by the methods and systems disclosed herein, such as the presence of a nucleic acid variant was detected in a sample, can be displayed directly in such a report. In some embodiments, only the presence or absence of a disease, such as cancer, is displayed in such a report.

The various steps of the methods disclosed herein, or steps carried out by the systems disclosed herein, may be carried out at the same or different times, in the same or different geographical locations, e.g., countries, and/or by the same or different people. In some embodiments, the report is communicated to a subject, for example, a subject who has cancer and has undergone testing by the methods and systems described herein, or to a healthcare professional, such as a physician treating the subject that has cancer.

This Summary is provided for purposes of illustrating some exemplary embodiments, so as to provide a basic understanding of some aspects of the subject matter described herein. Accordingly, it will be appreciated that the above-described features are examples and should not be construed to narrow the scope or spirit of the subject matter described herein in any way. Other features, aspects, and advantages of the subject matter described herein will become apparent from the following Detailed Description, Figures, and Claims.

### BRIEF DESCRIPTION OF THE FIGURES

FIG. 1 is a block diagram illustrating an example of a computer system in accordance with an embodiment of the present disclosure.

FIG. 2 is a flow diagram illustrating an example of a method for processing a sample in accordance with an embodiment of the present disclosure.

FIG. 3 is a drawing illustrating an example of motor protein adapter options in accordance with an embodiment of the present disclosure.

FIG. 4 is a drawing illustrating an example of a technique for processing a sample in accordance with an embodiment of the present disclosure.

FIG. 5 is a drawing illustrating an example of a technique for processing a sample using a two-ligation operation technique with additional A-tailing in accordance with an embodiment of the present disclosure.

FIG. 6 is a drawing illustrating an example of a technique for processing a sample using a two-ligation operation technique with additional A-tailing in accordance with an embodiment of the present disclosure.

FIG. 7 is a drawing illustrating an example of a technique for processing a sample by depleting or removing off-target regions in molecules of deoxyribonucleic acid (DNA) in accordance with an embodiment of the present disclosure.

FIG. 8 is a drawing illustrating an example of a technique for processing a sample by enriching the sample using a Cas9 protein or a restriction enzyme in accordance with an embodiment of the present disclosure.

FIG. 9 is a drawing illustrating an example of a technique for processing a sample by enriching the sample using a Cas9 protein or a restriction enzyme in accordance with an embodiment of the present disclosure.

FIG. 10 is a drawing illustrating an example of a technique for processing a sample by enriching the sample using a Cas9 protein or a restriction enzyme in accordance with an embodiment of the present disclosure.

FIG. 11 is a drawing illustrating an example of a technique for processing a sample by enriching the sample using a Cas9 protein or a restriction enzyme in accordance with an embodiment of the present disclosure.

FIG. 12 is a drawing illustrating an example of a technique for processing a sample using dead restriction enzyme (dRE) enrichment in accordance with an embodiment of the present disclosure.

FIG. 13 is a drawing illustrating an example of a technique for processing a sample using methyl-CpG-binding domain (MBD) protein enrichment in accordance with an embodiment of the present disclosure.

FIG. 14 is a drawing illustrating an example of a technique for processing a sample using catalytically inactive Cas9 (dCas9) enrichment of target regions in accordance with an embodiment of the present disclosure.

FIG. 15 is a drawing illustrating an example of a technique for processing a sample using chromatin immunoprecipitation (ChIP) enrichment of target regions in accordance with an embodiment of the present disclosure.

FIG. 16 is a drawing illustrating an example of a technique for processing a sample using a probe capture technique for an adapter in accordance with an embodiment of the present disclosure.

FIG. 17 is a drawing illustrating an example of a technique for processing a sample using a probe capture technique for an adapter in accordance with an embodiment of the present disclosure.

FIG. 18 is a drawing illustrating an example of a two-ligation technique for processing a sample in accordance with an embodiment of the present disclosure.

FIG. 19 is a drawing illustrating an example of a two-ligation technique for processing a sample in accordance with an embodiment of the present disclosure.

FIG. 20 is a drawing illustrating an example of a technique for processing a sample using a dumbbell-shaped adapter in accordance with an embodiment of the present disclosure.

FIG. 21 is a drawing illustrating an example of a technique for processing a sample using a hybrid MBD protein in accordance with an embodiment of the present disclosure.

FIG. 22 is a drawing illustrating an example of a two-ligation technique for processing a sample using tether enrichment probes in accordance with an embodiment of the present disclosure.

FIG. 23 is a drawing illustrating an example of a two-ligation technique for processing a sample in accordance with an embodiment of the present disclosure.

FIG. 24 is a drawing illustrating an example of a technique for processing a sample with surface-localized annealing in accordance with an embodiment of the present disclosure.

FIG. 25 is a drawing illustrating an example of a technique for processing a sample with surface-localized annealing in accordance with an embodiment of the present disclosure.

FIG. 26 is a drawing illustrating an example of a technique for processing a sample with surface-localized annealing in accordance with an embodiment of the present disclosure.

FIG. 27 is a drawing illustrating an example of a technique for processing a sample with surface-localized annealing in accordance with an embodiment of the present disclosure.

FIG. 28 is a drawing illustrating an example of a technique for processing a sample with surface-localized annealing in accordance with an embodiment of the present disclosure.

FIG. 29 is a block diagram illustrating an example of a computer in accordance with an embodiment of the present disclosure.

Note that like reference numerals refer to corresponding parts throughout the drawings. Moreover, multiple instances of the same part are designated by a common prefix separated from an instance number by a dash.

### DETAILED DESCRIPTION

An analysis technique for analyzing one or more target regions in molecules of deoxyribonucleic acid (DNA) in a sample (such as a tissue sample or cell-free DNA) is described. Notably, in this analysis technique, adapters are attached to ends of the molecules of DNA. Then, the molecules of DNA are selectively cut in one or more off-target regions in the molecules of DNA. Note that the selective cutting may use a Cas9 protein or a restriction enzyme. Moreover, the one or more off-target regions in the molecules of DNA are depleted or removed, where the off-target regions are different from the target regions. For example, the off-target regions may be digested or pulled out of solution. Next, after the depleting or removing operation, motor adapters are attached to a remainder of the molecules of DNA. Furthermore, the one or more target regions are analyzed by pulling or extracting the remainder of the molecules of DNA through an analysis membrane using the motor adapters.

By selectively cutting and depleting or removing the off-target regions, these analysis techniques may increase the accuracy of DNA results. Notably, the analysis techniques may increase the accuracy of DNA results for molecules in the target regions. Moreover, the analysis techniques may exclude the use of amplification of the one or more target regions. Instead, the processed sample may be analyzed using a nanopore. For example, the motor adapters may allow the remainder of the molecules of DNA to be pulled through the analysis membrane. This may allow accurate analysis of methylation of the target regions in the remainder of the molecules of DNA. Consequently, the analysis techniques may reduce the time and effort needed to analyze samples, and may reduce the incidence of incorrect results (such as false positives and false negatives) when analyzing samples. In the process, the analysis technique may increase confidence in the analysis results. Furthermore, the analysis techniques may facilitate early detection of disease (such as cancer), and may provide improved diagnosis, tracking of disease progression and treatment. Additionally, the analysis techniques may enable further understanding of a variety of types of cancer, and may facilitate the development of new treatments or therapeutic interventions. Consequently, the analysis techniques may reduce unnecessary or untimely therapeutic interventions, patient suffering and patient mortality. In some embodiments, more generally, the disclosed enrichment techniques may be used with a wide variety of sequencing or genomic analysis techniques.

In the discussion that follows, the analysis techniques are used to determine analysis results for samples that include or correspond to a wide variety of genetic molecules or information, including: DNA (such as double-stranded or single-stranded when there is information available to establish stand bias), cell-free nucleic acid, ribonucleic acid (RNA), epigenetic information, gene expression or transcriptional state information, protein information, etc. In the discussion that follows, DNA corresponding to at least a portion of an individual's genome is used as an illustrative example.

Moreover, in order for the present disclosure to be more readily understood, certain terms are first defined below. Additional definitions for the following terms and other terms may be set forth through the specification. If a definition of a term set forth below is inconsistent with a definition in an application or patent that is incorporated by reference, the definition set forth in this application should be used to understand the meaning of the term

As used in this specification and the appended claims, the singular forms ‘a’, ‘an’, and ‘the’ include plural references unless the context clearly dictates otherwise. Thus, e.g., a reference to ‘a method’ includes one or more methods, and/or operations of the type described herein and/or which will become apparent to those persons of ordinary skill in the art upon reading this disclosure and so forth.

Moreover, ‘optional’ or ‘optionally’ means that the subsequently described event or circumstance may or may not occur, and that the description includes cases where said event or circumstance occurs and cases where it does not.

Furthermore, throughout the description and claims of this specification, the word ‘comprise’ and variations of the word, such as ‘comprising’ and ‘comprises,’ means ‘including but not limited to,’ and is not intended to exclude, for example, other components, integers or operations. ‘Exemplary’ means ‘an example of’ and is not intended to convey an indication of a preferred or ideal configuration. ‘Such as’ is not used in a restrictive sense, but for explanatory purposes.

It is also to be understood that the terminology used herein is for the purpose of describing particular embodiments only, and is not intended to be limiting. Furthermore, unless defined otherwise, all technical and scientific terms used herein have the same meaning as commonly understood by one of ordinary skill in the art to which this disclosure pertains. In describing and claiming the methods, computer-readable media, and systems, the following terminology, and grammatical variants thereof, will be used in accordance with the definitions set forth below.

About: As used herein, ‘about’ or ‘approximately’ as applied to one or more values or elements of interest, refers to a value or element that is similar to a stated reference value or element. In certain embodiments, the term ‘about’ or ‘approximately’ refers to a range of values or elements that falls within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value or element unless otherwise stated or otherwise evident from the context (except where such number would exceed 100% of a possible value or element).

Adapter: As used herein, ‘adapter’ refers to a short nucleic acid (e.g., less than about 500 nucleotides, less than about 100 nucleotides, or less than about 50 nucleotides in length) that is typically at least partially double-stranded and used to link to either or both ends of a given sample nucleic acid molecule. Adapters can include nucleic acid primer binding sites to permit amplification of a nucleic acid molecule flanked by adapters at both ends, and/or a sequencing primer binding site, including primer binding sites for sequencing applications, such as various next-generation sequencing (NGS) applications. Adapters can also include binding sites for capture probes, such as an oligonucleotide attached to a flow cell support or the like. Adapters can also include a nucleic acid tag as described herein. Nucleic acid tags are typically positioned relative to amplification primer and sequencing primer binding sites, such that a nucleic acid tag is included in amplicons and sequence reads of a given nucleic acid molecule. The same or different adapters can be linked to the respective ends of a nucleic acid molecule. In some embodiments, an adapter of the same sequence is linked to the respective ends of the nucleic acid molecule except that the nucleic acid tag differs. In some embodiments, the adapter is a Y-shaped adapter in which one end is blunt ended or tailed as described herein, for joining to a nucleic acid molecule, which is also blunt ended or tailed with one or more complementary nucleotides. In still other example embodiments, an adapter is a bell-shaped adapter that includes a blunt or tailed end for joining to a nucleic acid molecule to be analyzed. Other examples of adapters include T-tailed and C-tailed adapters.

Amplify: As used herein, ‘amplify’ or ‘amplification’ in the context of nucleic acids refers to the production of multiple copies of a polynucleotide, or a portion of the polynucleotide, typically starting from a small amount of the polynucleotide (e.g., a single polynucleotide molecule), where the amplification products or amplicons are generally detectable. Amplification of polynucleotides encompasses a variety of chemical and enzymatic processes.

Barcode: As used herein, ‘barcode’ or ‘molecular barcode’ in the context of nucleic acids refers to a nucleic acid molecule including a sequence that can serve as a molecular identifier. For example, individual ‘barcode’ sequences are typically added to each DNA fragment during next-generation sequencing library preparation so that each read can be identified and sorted before the final data analysis. In some embodiments, the one or more molecular barcodes is at least 2, at least 4, at least 5, at least 6, at least 8, at least 10, at least 15 or at least 20 nucleotides in length. In some embodiments, the polynucleotides of the sample are tagged with at least 5, at least 10, at least 15, at least 20, at least 50, at least 100, at least 500, at least 1000, at least 5000, at least 10,000, at least 50,000 or at least 100,000 different tags/molecular barcodes.

Cancer Type: As used herein, ‘cancer type’ refers to a type or subtype of cancer defined, e.g., by histopathology. Cancer type can be defined by any conventional criterion, such as on the basis of occurrence in a given tissue (e.g., blood cancers, central nervous system or CNS, brain cancers, lung cancers such as small cell and non-small cell, skin cancers, nose cancers, throat cancers, liver cancers, bone cancers, lymphomas, pancreatic cancers, bowel cancers, rectal cancers, thyroid cancers, bladder cancers, kidney cancers, mouth cancers, stomach cancers, breast cancers, prostate cancers, ovarian cancers, lung cancers, intestinal cancers, soft tissue cancers, neuroendocrine cancers, gastroesophageal cancers, head and neck cancers, gynecological cancers, colorectal cancers, urothelial cancers, solid state cancers, heterogeneous cancers, homogenous cancers, or another cancer type), unknown primary origin and the like, and/or of the same cell lineage (e.g., carcinoma, sarcoma, lymphoma, cholangiocarcinoma, leukemia, mesothelioma, melanoma, or glioblastoma), and/or cancers exhibiting cancer markers, such as: Her2, CA15-3, CA19-9, CA-125, CEA, AFP, PSA, HCG, hormone receptor and NMP-22. Cancers can also be classified by stage (e.g., stage 1, 2, 3, or 4) and whether of primary or secondary origin.

Cell-Free Nucleic Acid: As used herein, ‘cell-free nucleic acid’ refers to nucleic acids not contained within or otherwise bound to a cell or, in some embodiments, nucleic acids remaining in a sample following the removal of intact cells. Notably, ‘cell-free nucleic acid’ is ‘cell free’ at the point of isolation from a subject. Therefore, cell-free nucleic acid may not encompass or may be different from isolated cellular DNA. Cell-free nucleic acids can include, e.g., all non-encapsulated nucleic acids sourced from a bodily fluid (e.g., blood, plasma, serum, urine, cerebrospinal fluid or CSF, etc.) from a subject. Cell-free nucleic acids include DNA (cfDNA), RNA (cfRNA), and hybrids thereof, including genomic DNA, mitochondrial DNA, circulating DNA, siRNA, miRNA, circulating RNA (cRNA), tRNA, rRNA, small nucleolar RNA (snoRNA), Piwi-interacting RNA (piRNA), long non-coding RNA (long ncRNA), and/or fragments of any of these. Cell-free nucleic acids can be double-stranded, single-stranded, or a hybrid thereof. A cell-free nucleic acid can be released into bodily fluid through secretion or cell-death processes, e.g., cellular necrosis, apoptosis, or the like. Some cell-free nucleic acids are released into bodily fluid from cancer cells, e.g., circulating tumor DNA (ctDNA). Others are released from healthy cells. CtDNA can be non-encapsulated tumor-derived fragmented DNA. Another example of cell-free nucleic acids is fetal DNA circulating freely in the maternal blood stream, also called cell-free fetal DNA (cffDNA). A cell-free nucleic acid can have one or more epigenetic modifications, e.g., a cell-free nucleic acid can be (or a histone associated with the cell-free nucleic acid can be) acetylated, 5-methylated, ubiquitylated, phosphorylated, sumoylated, ribosylated, and/or citrullinated.

Cellular Nucleic Acids: As used herein, ‘cellular nucleic acids’ means nucleic acids that are disposed within one or more cells from which the nucleic acids have originated, at least at the point a sample is taken or collected from a subject, even if those nucleic acids are subsequently removed (e.g., via cell lysis) as part of a given analytical process.

Deoxyribonucleic Acid or Ribonucleic Acid: As used herein, ‘deoxyribonucleic acid’ or ‘DNA’ refers to a natural or modified nucleotide which has a hydrogen group at the 2′-position of the sugar moiety. DNA typically includes a chain of nucleotides including four types of nucleotide bases; adenine (A), thymine (T), cytosine (C), and guanine (G). As used herein, ‘ribonucleic acid’ or ‘RNA’ refers to a natural or modified nucleotide which has a hydroxyl group at the 2′-position of the sugar moiety. RNA typically includes a chain of nucleotides including four types of nucleotide bases; A, uracil (U), G, and C. As used herein, the term ‘nucleotide’ refers to a natural nucleotide or a modified nucleotide. Certain pairs of nucleotides specifically bind to one another in a complementary fashion (called complementary base pairing). In DNA, adenine (A) pairs with thymine (T) and cytosine (C) pairs with guanine (G). In RNA, adenine (A) pairs with uracil (U) and cytosine (C) pairs with guanine (G). When a first nucleic acid strand binds to a second nucleic acid strand made up of nucleotides that are complementary to those in the first strand, the two strands bind to form a double strand. As used herein, ‘nucleic acid sequencing data,’ ‘nucleic acid sequencing information,’ ‘sequence information,’ ‘nucleic acid sequence,’ ‘nucleotide sequence,’ ‘genomic sequence,’ ‘genetic sequence,’ ‘fragment sequence,’ or ‘nucleic acid sequencing read’ denotes any information or data that is indicative of the order and identity of the nucleotide bases (e.g., adenine, guanine, cytosine, and thymine or uracil) in a molecule (e.g., a whole genome, whole transcriptome, exome, oligonucleotide, polynucleotide, or fragment) of a nucleic acid such as DNA or RNA. It should be understood that the present teachings contemplate sequence information obtained using all available varieties of techniques, platforms or technologies, including, but not limited to: capillary electrophoresis, microarrays, ligation-based systems, polymerase-based systems, hybridization-based systems, direct or indirect nucleotide identification systems, pyrosequencing, ion- or pH-based detection systems, and electronic signature-based systems.

DNA-like RNA (dRNA): As used herein, ‘dRNA’ refers to RNA molecules that are not included in the ribosomal RNA (rRNA) and transfer RNA (tRNA) classes. Much of the dRNA is of high molecular weight, short half-life, and never leaves the nucleus.

Chromatin: As used herein, ‘chromatin’ refers to a mixture of DNA and proteins that form the chromosomes found in the cells of humans and other higher organisms. Many of the proteins (notably, histones) package the massive amount of DNA in a genome into a highly compact form that can fit in the cell nucleus.

Germline Mutation: As used herein, the terms ‘germline mutation’ or ‘germline variation’ are used interchangeably and refer to an inherited mutation (or not one arising post-conception). Germline mutations may be the only mutations that can be passed on to the offspring and may be present in every somatic cell and germline cell in the offspring.

Indel: As used herein, ‘indel’ refers to a mutation that involves the insertion or deletion of nucleotides in the genome of a subject.

Methyl-CpG-binding domain (MBD) protein: As used herein ‘MBD protein’ are a family of nuclear proteins closely related to DNA methylation that read and bind methylated DNA, recruit additional chromatin remodeling proteins to modify histones, and repress gene expression.

Minor Allele Frequency: As used herein, ‘minor allele frequency’ or ‘MAF’ refers to the frequency at which minor alleles (e.g., not the most common allele) occurs in a given population of nucleic acids, such as a sample obtained from a subject. Genetic variants at a low minor allele frequency typically have a relatively low frequency of presence in a sample.

Mutant Allele Fraction: As used herein, ‘mutant allele fraction’ or ‘mutation dose’ refers to the fraction of nucleic acid molecules harboring an allelic alteration or mutation at a given genomic position/locus in a given sample. The mutant allele fraction is generally expressed as a fraction or a percentage. For example, a mutant allele fraction of a somatic variant may be less than 0.15.

Mutation: As used herein, ‘mutation’ refers to a variation from a known reference sequence and includes mutations such as, e.g., single nucleotide variants or SNVs, and insertions or deletions or indels. A mutation can be a germline or somatic mutation. In some embodiments, a reference sequence for purposes of comparison is a wildtype genomic sequence of the species of the subject providing a test sample, typically the human genome.

Neoplasm: As used herein, the terms ‘neoplasm’ and ‘tumor’ are used interchangeably. They refer to abnormal growth of cells in a subject. A neoplasm or tumor can be benign, potentially malignant, or malignant. A malignant tumor is a referred to as a cancer or a cancerous tumor.

Next Generation Sequencing: As used herein, ‘next generation sequencing’ or ‘NGS’ refers to sequencing technologies having increased throughput as compared to traditional Sanger- and capillary electrophoresis-based approaches, e.g., with the ability to generate hundreds of thousands of relatively small sequence reads at a time. Some examples of next generation sequencing techniques include, but are not limited to, sequencing by synthesis, sequencing by ligation, and sequencing by hybridization.

Nucleic Acid Tag: As used herein, ‘nucleic acid tag’ refers to a short nucleic acid (e.g., less than about 500 nucleotides, about 100 nucleotides, about 50 nucleotides, or about 10 nucleotides in length), used to distinguish nucleic acids from different samples (e.g., representing a sample index), or different nucleic acid molecules in the same sample (e.g., representing a molecular barcode), of different types, or which have undergone different processing. The nucleic acid tag includes a predetermined, fixed, non-random, random or semi-random oligonucleotide sequence. Such nucleic acid tags may be used to label different nucleic acid molecules or different nucleic acid samples or sub-samples. Nucleic acid tags can be single-stranded, double-stranded, or at least partially double-stranded. Nucleic acid tags optionally have the same length or varied lengths. Nucleic acid tags can also include double-stranded molecules having one or more blunt-ends, include 5′ or 3′ single-stranded regions (e.g., an overhang), and/or include one or more other single-stranded regions at other locations within a given molecule. Nucleic acid tags can be attached to one end or to both ends of the other nucleic acids (e.g., sample nucleic acids to be amplified and/or sequenced). Nucleic acid tags can be decoded to reveal information such as the sample of origin, form, or processing of a given nucleic acid. For example, nucleic acid tags can also be used to enable pooling and/or parallel processing of multiple samples including nucleic acids bearing different molecular barcodes and/or sample indexes in which the nucleic acids are subsequently being deconvolved by detecting (e.g., reading) the nucleic acid tags. Nucleic acid tags can also be referred to as identifiers (e.g., molecular identifier or sample identifier). Additionally, or alternatively, nucleic acid tags can be used as molecular barcodes (e.g., to distinguish between different molecules or amplicons of different parent molecules in the same sample or sub-sample). This includes, e.g., uniquely tagging different nucleic acid molecules in a given sample, or non-uniquely tagging such molecules. In the case of non-unique tagging applications, a limited number of tags (such as molecular barcodes) may be used to tag the nucleic acid molecules such that different molecules can be distinguished based on their endogenous sequence information (for example, start and/or stop positions where they map to a selected reference genome, a sub-sequence of one or both ends of a sequence, and/or length of a sequence) in combination with at least one molecular barcode. Typically, a sufficient number of different molecular barcodes are used such that there is a low probability (e.g., less than about a 10%, less than about a 5%, less than about a 1%, or less than about a 0.1% chance) that any two molecules may have the same endogenous sequence information (e.g., start and/or stop positions, subsequences of one or both ends of a sequence, and/or lengths) and also have the same molecular barcode.

Polynucleotide: As used herein, ‘polynucleotide,’ ‘nucleic acid,’ ‘nucleic acid molecule,’ or ‘oligonucleotide’ refers to a linear polymer of nucleosides (including deoxyribonucleosides, ribonucleosides, or analogs thereof) joined by inter-nucleosidic linkages. Typically, a polynucleotide includes at least three nucleosides. Oligonucleotides often range in size from a few monomeric units, e.g., 3-4, to hundreds of monomeric units. Whenever a polynucleotide is represented by a sequence of letters, such as ‘ATGCCTG,’ it will be understood that the nucleotides are in 5′→3′ order from left to right and that in the case of DNA, ‘A’ denotes deoxyadenosine, ‘C’ denotes deoxycytidine, ‘G’ denotes deoxyguanosine, and ‘T’ denotes deoxythymidine, unless otherwise noted. The letters A, C, G, and T may be used to refer to the bases themselves, to nucleosides, or to nucleotides including the bases, as is standard in the art.

Reference Sequence: As used herein, ‘reference sequence’ refers to a known sequence used for purposes of comparison with experimentally determined sequences. For example, a known sequence can be an entire genome, a chromosome, or any segment thereof. A reference typically includes at least about 20, at least about 50, at least about 100, at least about 200, at least about 250, at least about 300, at least about 350, at least about 400, at least about 450, at least about 500, at least about 1000, or more than 1000 nucleotides. A reference sequence can align with a single contiguous sequence of a genome or chromosome or can include non-contiguous segments that align with different regions of a genome or chromosome. Examples of reference sequences include, e.g., human genomes, such as, hG19 and hG38.

Restriction enzyme: As used herein, a ‘restriction enzyme’ is a protein isolated from bacteria that cleaves DNA sequences at sequence-specific sites, producing DNA fragments with a known sequence at each end. An example of a restriction enzyme is Cas9. Cas9 enzymes include an HNH domain that cleaves the DNA strand complementary to the guide RNA sequence (or target strand), and a RuvC nuclease domain that cleaves the noncomplementary strand (or non-target strand), yielding double strand DNA breaks (DSBs). By mutating both the HNH and RuvC domains of Cas9, the DNA-cleaving activity of Cas9 can be blocked. This catalytically inactive form of Cas9 is known as ‘dead Cas9’ (dCas9). This variant still binds to targeted DNA when directed there by a guide RNA, but it does not cleave the DNA. In the present discussion, such a restriction protein is more generally referred to as a ‘dead restriction enzyme (dRE).’

Sample: As used herein, ‘sample’ means anything capable of being analyzed by the methods and/or systems disclosed herein. For example, a sample may include a normal tissue sample or a tissue sample associated with a type of disease, such as a type of cancer.

Sequencing: As used herein, ‘sequencing’ refers to any of a number of technologies used to determine the sequence (e.g., the identity and order of monomer units) of a biomolecule, e.g., a nucleic acid such as DNA or RNA. Examples of sequencing methods include, but are not limited to, targeted sequencing, single molecule real-time sequencing, exon or exome sequencing, intron sequencing, electron microscopy-based sequencing, panel sequencing, transistor-mediated sequencing, direct sequencing, random shotgun sequencing, Sanger dideoxy termination sequencing, whole-genome sequencing, sequencing by hybridization, pyrosequencing, capillary electrophoresis, duplex sequencing, cycle sequencing, single-base extension sequencing, solid-phase sequencing, high-throughput sequencing, massively parallel signature sequencing, emulsion polymerase chain reaction (PCR), co-amplification at lower denaturation temperature-PCR (COLD-PCR), multiplex PCR, sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, nanopore sequencing, 454 sequencing, Solexa Genome Analyzer sequencing (from Illumina of San Diego, California), SOLiD™ sequencing (from Life Technologies, a division of Thermo Fisher Scientific of Waltham, Massachusetts), MS-PET sequencing, and a combination thereof. In some embodiments, sequencing can be performer by a gene analyzer such as, e.g., gene analyzers commercially available from Illumina, Inc., Pacific Biosciences, Inc. (of Menlo Park, California), or Applied Biosystems/Thermo Fisher Scientific, among many others. Note that, in some embodiments, sequencing may include determining a base identity at a single position or loci.

Sequence Information: As used herein, ‘sequence information’ in the context of a nucleic acid polymer means the order and identity of monomer units (e.g., nucleotides, etc.) in that polymer.

Single Nucleotide Polymorphism: As used herein, the terms ‘single nucleotide polymorphism’ or ‘SNP’ are used interchangeably. They refer to a variation in a single nucleotide that occurs at a specific position in the genome, where each variation is present to some appreciable degree of frequency within a population (e.g., greater than about 1%).

Single Nucleotide Variant: As used herein, ‘single nucleotide variant’ or ‘SNV’ means a mutation or variation in a single nucleotide that occurs at a specific position in the genome.

Somatic Mutation: As used herein, the terms ‘somatic mutation’ or ‘somatic variation’ are used interchangeably. They refer to a mutation in the genome that occurs after conception. Somatic mutations can occur in any cell of the body except germ cells and accordingly, are not passed on to progeny.

Subject: As used herein, ‘subject’ refers to an animal, such as a mammalian species (e.g., human) or avian (e.g., bird) species, or other organism, such as a plant. More specifically, a subject can be a vertebrate, e.g., a mammal such as a mouse, a primate, a simian or a human. Animals include farm animals (e.g., production cattle, dairy cattle, poultry, horses, pigs, and the like), sport animals, and companion animals (e.g., pets or support animals). A subject can be a healthy individual, an individual that has or is suspected of having a disease or a predisposition to the disease, or an individual in need of therapy or suspected of needing therapy. The terms ‘individual’ or ‘patient’ are intended to be interchangeable with ‘subject.’

For example, a subject can be an individual who has been diagnosed with having a cancer, is going to receive a cancer therapy, and/or has received at least one cancer therapy. The subject can be in remission of a cancer. As another example, the subject can be an individual who is diagnosed of having an autoimmune disease. As another example, the subject can be a female individual who is pregnant or who is planning on getting pregnant, who may have been diagnosed of or suspected of having a disease, e.g., a cancer, an auto-immune disease.

Substantially identical: As used herein, the term ‘substantially identical’ refers to two different entities that are 99.9% identical, at least 95% identical, at least 90% identical, at least 85% identical, at least 80% identical, at least 75% identical, at least 70% identical, at least 60% identical or at least 50% identical. In cases where the entity is the molecular barcode, then the term ‘substantially identical’ refers to two different molecular barcodes that have a Hamming distance or edit distance of less than 2, less than 3, less than 4, less than 5, less than 6, less than 7 or less than 8. In cases where the entity is the beginning region or end region, then the term ‘substantially identical’ refers to two different regions that are within 1 bp, within 2 bp, within 3 bp, within 4 bp, within 5 bp, within 6 bp, within 7 bp, within 8 bp, within 9 bp, within 10 bp, within 11 bp, within 15 bp, within 20 bp or within 25 bp. In cases where the entity is the length of the polynucleotide, then the term ‘substantially identical’ refers to two different lengths that are within 1 bp, within 2 bp, within 3 bp, within 4 bp, within 5 bp, within 6 bp, within 7 bp, within 8 bp, within 9 bp, within 10 bp, within 11 bp, within 15 bp, within 20 bp, within 25 bp, within 30 bp, within 40 bp or within 50 bp.

Threshold: As used herein, ‘threshold’ refers to a predetermined value used to characterize experimentally determined values of the same parameter for different samples depending on their relation to the threshold. For example, the threshold for the p-value can refer to any predetermined value between 0 and 1 and is used to identify the origin of a nucleic acid variant.

Variant: As used herein, a ‘variant’ can be referred to as an allele. A variant is usually presented at a frequency of 50% (0.5) or 100% (1), depending on whether the allele is heterozygous or homozygous. For example, germline variants are inherited and usually have a frequency of 0.5 or 1. Somatic variants, however, are acquired variants and usually have a frequency of less than about 0.5. Major and minor alleles of a genetic locus refer to nucleic acids harboring the locus in which the locus is occupied by a nucleotide of a reference sequence, and a variant nucleotide different than the reference sequence respectively. Measurements at a locus can take the form of allelic fractions (AFs), which measure the frequency with which an allele is observed in a sample.

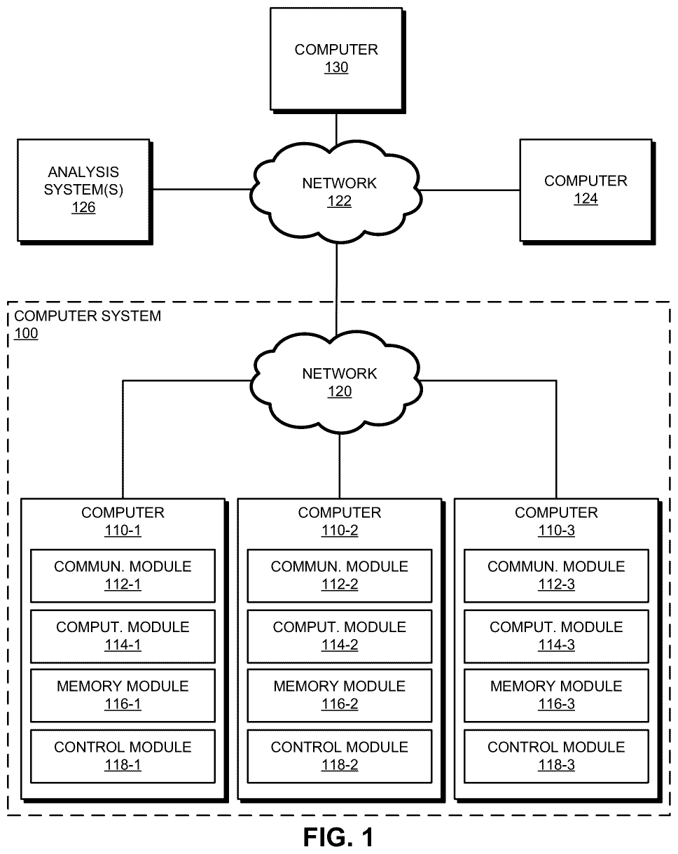

We now describe embodiments of the analysis techniques. FIG. 1 presents a block diagram illustrating an example of a computer system 100. This computer system may include one or more computers 110. These computers may include: communication modules 112, computation modules 114, memory modules 116, and optional control modules 118. Note that a given module or engine may be implemented in hardware and/or in software.

Communication modules 112 may communicate frames or packets with data or information (such as measurement results or control instructions) between computers 110 via a network 120 (such as the Internet and/or an intranet). For example, this communication may use a wired communication protocol, such as an Institute of Electrical and Electronics Engineers (IEEE) 802.3 standard (which is sometimes referred to as ‘Ethernet’) and/or another type of wired interface. Alternatively or additionally, communication modules 112 may communicate the data or the information using a wireless communication protocol, such as: an IEEE 802.11 standard (which is sometimes referred to as ‘Wi-Fi’, from the Wi-Fi Alliance of Austin, Texas), Bluetooth (from the Bluetooth Special Interest Group of Kirkland, Washington), a third generation or 3G communication protocol, a fourth generation or 4G communication protocol, e.g., Long Term Evolution or LTE (from the 3rd Generation Partnership Project of Sophia Antipolis, Valbonne, France), LTE Advanced (LTE-A), a fifth generation or 5G communication protocol, other present or future developed advanced cellular communication protocol, or another type of wireless interface. For example, an IEEE 802.11 standard may include one or more of: IEEE 802.11a, IEEE 802.11b, IEEE 802.11g, IEEE 802.11-2007, IEEE 802.11n, IEEE 802.11-2012, IEEE 802.11-2016, IEEE 802.11ac, IEEE 802.11ax, IEEE 802.11ba, IEEE 802.11be, IEEE 802.11bn, or other present or future developed IEEE 802.11 technologies.

In the described embodiments, processing a packet or a frame in a given one of computers 110 (such as computer 110-1) may include: receiving the signals with a packet or the frame; decoding/extracting the packet or the frame from the received signals to acquire the packet or the frame; and processing the packet or the frame to determine information contained in the payload of the packet or the frame. Note that the communication in FIG. 1 may be characterized by a variety of performance metrics, such as: a data rate for successful communication (which is sometimes referred to as ‘throughput’), an error rate (such as a retry or resend rate), a mean squared error of equalized signals relative to an equalization target, intersymbol interference, multipath interference, a signal-to-noise ratio, a width of an eye pattern, a ratio of number of bytes successfully communicated during a time interval (such as 1-10 s) to an estimated maximum number of bytes that can be communicated in the time interval (the latter of which is sometimes referred to as the ‘capacity’ of a communication channel or link), and/or a ratio of an actual data rate to an estimated data rate (which is sometimes referred to as ‘utilization’). Note that wireless communication between components in FIG. 1 uses one or more bands of frequencies, such as: 900 MHZ, 2.4 GHz, 5 GHZ, 6 GHZ, 60 GHz, the Citizens Broadband Radio Spectrum or CBRS (e.g., a frequency band near 3.5 GHZ), and/or a band of frequencies used by LTE or another cellular-telephone communication protocol or a data communication protocol. In some embodiments, the communication between the components may use multi-user transmission (such as orthogonal frequency division multiple access or OFDMA) and/or multiple-input multiple-output (MIMO).

Moreover, computation modules 114 may perform calculations using: one or more microprocessors, ASICs, microcontrollers, programmable-logic devices, GPUs and/or one or more digital signal processors (DSPs). Note that a given computation component is sometimes referred to as a ‘computation device’.

Furthermore, memory modules 116 may access stored data or information in memory that local in computer system 100 and/or that is remotely located from computer system 100. Notably, in some embodiments, one or more of memory modules 116 may access stored measurement results in the local memory, such as MRI data for one or more individuals (which, for multiple individuals, may include cases and controls or disease and healthy populations). Alternatively or additionally, in other embodiments, one or more memory modules 116 may access, via one or more of communication modules 112, stored measurement results in the remote memory in computer 124, e.g., via network 120 and network 122. Note that network 122 may include: the Internet and/or an intranet. In some embodiments, the measurement results are received from one or more analysis systems 126 (such as PCR, a whole genome sequencer or a partial genome sequencer, e.g., a whole exome sequencer or, more generally, a gene sequencer that uses: a gene sequencing panel, Sanger sequencing, capillary electrophoresis and fragment analysis, microarrays, ligation-based systems, polymerase-based systems, hybridization-based systems, direct or indirect nucleotide identification systems, pyrosequencing, ion- or pH-based detection systems, electronic signature-based systems, next generation sequencing, long-read genetic sequencing, sequencing based on nanopore technology, and/or another sequencing technique) via network 120 and network 122 and one or more of communication modules 112. Thus, in some embodiments at least some of the measurement results may have been received previously and may be stored in memory, while in other embodiments at least some of the measurement results may be received in real-time from the one or more analysis systems 126.

While FIG. 1 illustrates computer system 100 at a particular location, in other embodiments at least a portion of computer system 100 is implemented at more than one location. Thus, in some embodiments, computer system 100 is implemented in a centralized manner, while in other embodiments at least a portion of computer system 100 is implemented in a distributed manner (such as using cloud-computing resources). For example, in some embodiments, the one or more analysis systems 126 may include local hardware and/or software that performs at least some of the operations in the analysis techniques. This remote processing may reduce the amount of data that is communicated via network 120 and network 122. In addition, the remote processing may anonymize the measurement results that are communicated to and analyzed by computer system 100. This capability may help ensure computer system 100 is compatible and compliant with regulations, such as the Health Insurance Portability and Accountability Act, e.g., by removing or obfuscating protected health information in the measurement results.

Although we describe the computation environment shown in FIG. 1 as an example, in alternative embodiments, different numbers or types of components may be present in computer system 100. For example, some embodiments may include more or fewer components, a different component, and/or components may be combined into a single component, and/or a single component may be divided into two or more components.

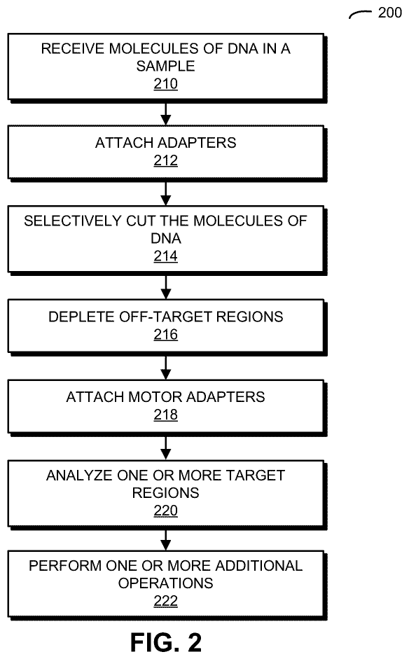

As discussed previously, it can be difficult to accurately analyze molecules of DNA, such as fragments of DNA or cell-free DNA. Moreover, as described further below with reference to FIGS. 2-28, in order to address these challenges computer system 100 may perform the analysis techniques or instructions for the analysis techniques. Notably, during the analysis techniques, one or more of optional control modules 118 may divide the analysis among computers 110. Then, a given computer (such as computer 110-1) may perform at least a designated portion of the analysis. Notably, computation module 114-1 may receive (e.g., access) information (e.g., using memory module 116-1) specifying molecules or genetic molecules (such as at least portions of DNA) from a sample that is associated with an individual. The information may include genotype information, such as: nucleotides as a function of location on at least a strand or in the DNA; mutations or variants as a function of location on at least a strand or in the DNA (such as an SNV, a CNV, a fusion, an insertion, a deletion and/or an epigenetic change); alleles as a function of location on at least a strand or in the DNA; epigenetic information as a function of on at least a strand or in the DNA; genetic information corresponding to molecules of DNA; and/or another type of genomic information as a function of location on at least a strand or in the DNA. Note that the aforementioned locations may be at least a subset of the loci in the DNA. Thus, the locations may include one or more loci in the DNA. In some embodiments, the molecules of DNA may include cell-free DNA having lengths less than 200 bases.

Then, computation module 114-1 may perform operations in the analysis techniques. Notably, the analysis techniques may include: providing (e.g., via communication module 112-1 to analysis system 126) an instruction to attach adapters to ends of the molecules of DNA. Moreover, the analysis techniques may include: providing (e.g., via communication module 112-1 to analysis system 126) a second instruction to selectively cut the molecules of DNA in one or more off-target regions (e.g., at least a subset of the fragment ends in the molecules of DNA), where a given cut results in fragment ends in the molecules of DNA. Note that the adapters may prevent the selective cutting. For example, the adapters may include ligation adapters, and a given off-target region may be separated from a given target region by a given ligation adapter. In some embodiments, the selective cutting uses a Cas9 protein or a restriction enzyme.

Next, the analysis techniques may include: providing (e.g., via communication module 112-1 to analysis system 126) a third instruction to deplete or remove the off-target regions in the molecules of DNA, where the off-target regions in the molecules of DNA are different from target regions in the molecules of DNA having methylation or binding states. Notably, the depletion or removal may include: digesting the off-target regions in the molecules of DNA; or pulling or extracting out of solution the digested off-target regions in the molecules of DNA. For example, the digestion may include exonuclease digestion. Alternatively or additionally, the pulling or extracting out may use magnetic beads, such as streptavidin magnetic beads.

Moreover, following the depleting or removing operation, the analysis techniques may include: providing (e.g., via communication module 112-1 to analysis system 126) a fourth instruction to attach motor adapters to locations on a remainder of the molecules of DNA. Furthermore, the analysis techniques may include: analyzing (e.g., via communication module 112-1 and analysis system 126) the one or more target regions by pulling the remainder of the molecules of DNA through an analysis membrane (such as a nanopore membrane) using the motor adapters. Note that the analysis may exclude amplification of the one or more target regions. Additionally, the analysis may include determining the molecules of DNA in the one or more target regions. For example, the analysis may include determining the methylation states or the binding states of the one or more target regions.

Computation module 114-1 may store results of the analysis, e.g., in memory module 116-1. Alternatively or additionally, computation module 114-1 may provide results of the analysis. For example, computation module 114-1 may display the results (e.g., on a display associated with computer system 100) and/or may instruction communication module 112-1 to provide the results, e.g., to computer 124 or computer 130, which may be associated with a physician (such as a pathologist) or healthcare provider of the individual. In some embodiments, the results may be included in a report about an individual associated with the sample (such a computer-aided diagnosis report with feedback, such as a quality metric, call variants, a cancer classification, a treatment recommendation, etc.). Note that the results may include: a quality metric, mutations or call variants, a cancer classification, such as an indication that a type of cancer is present in the sample (e.g., that a clinical variant has been detected), a treatment recommendation (such as a recommendation for radiation or chemotherapy, a type of chemotherapy, etc.) based at least in part on the results.

In some embodiments, may be performed using a pretrained predictive model, such as a classifier or a regression model. Notably, the analysis techniques may include inputting the results of prior operations in the analysis to the pretrained predictive model. The pretrained predictive model may output the results of the analysis at or corresponding to one or more locations in the DNA. In general, the pretrained predictive model may include a machine-learning model or a neural network, which was previously trained using a training dataset. Furthermore, the calling of variants may be performed using the same or a different pretrained model, such as a second machine-learning model or a second neural network, which was previously trained using a second training dataset. In some embodiments, the pretrained predictive model or the second pretrained predictive model may use information specifying the analysis technique (such as a type of DNA probe) as an input. Note that a given neural network may include or combine: one or more convolutional layers, one or more residual layers and one or more dense or fully connected layers, and where a given node in a given layer in the given neural network may include an activation function, such as: a rectified linear activation function or ReLU, a leaky ReLU, an exponential linear unit or ELU activation function, a parametric ReLU, a tanh activation function, and/or a sigmoid activation function.

In these ways, computer system 100 may automatically and accurately analyze target regions in the remainder of the molecules in the DNA. For example, the cutting and depleting or removing of the off-target regions, and the selective attachment and use of motor adapters may allow accurate analysis of methylation of the target regions in the remainder of the molecules of DNA. Consequently, the analysis techniques may reduce the time and effort needed to analyze samples, and may reduce the incidence of incorrect results (such as false positives and false negatives) when analyzing samples. In the process, the analysis technique may increase confidence in the analysis results. Furthermore, the analysis techniques may facilitate early detection of disease (such as cancer), and may provide improved diagnosis, tracking of disease progression and treatment. Additionally, the analysis techniques may enable further understanding of a variety of types of cancer, and may facilitate the development of new treatments or therapeutic interventions. Consequently, the analysis techniques may reduce unnecessary or untimely therapeutic interventions, patient suffering and patient mortality.

We now describe embodiments of the method. FIG. 2 presents a flow diagram illustrating an example of a method 200 for processing a sample, which may be performed by a computer system (such as computer system 100 in FIG. 1). During method 200, molecules of DNA in the sample may be received (operation 210) or collected, or may receive information describing the molecules of DNA. Then, adapters may be attached (operation 212) to ends of the molecules of DNA. Moreover, the molecules of DNA may be selectively cut (operation 214) in one or more off-target regions, where a given cut results in fragment ends in the molecules of DNA. Next, the off-target regions in the molecules of DNA may be depleted or removed (operation 216), where the off-target regions in the molecules of DNA are different from target regions in the molecules of DNA having methylation or binding states. Moreover, following the depleting or removing operation, motor adapters may be attached (operation 218) to locations on a remainder of the molecules of DNA. Furthermore, the one or more target regions may be analyzed (operation 220) by pulling or extracting the remainder of the molecules of DNA through an analysis membrane using the motor adapters.

Note that the adapters may prevent the selective cutting. For example, the adapters may include ligation adapters, and a given off-target region may be separated from a given target region by a given ligation adapter. In some embodiments, the selective cutting uses a Cas9 protein or a restriction enzyme.

Moreover, the depletion or removal (operation 216) may include: digesting the off-target regions in the molecules of DNA; or pulling or extracting out of solution the digested off-target regions in the molecules of DNA. For example, the digestion may include exonuclease digestion. Alternatively or additionally, the pulling or extracting out may use magnetic beads, such as streptavidin magnetic beads.

Furthermore, the off-target regions may include at least a subset of the fragment ends in the molecules of DNA.

Additionally, the analysis membrane may include a nanopore membrane.

In some embodiments, the molecules of DNA may include cell-free DNA having lengths less than 200 bases.

Moreover, the analysis (operation 220) may include determining the molecules of DNA in the one or more target regions. For example, the analysis (operation 220) may include determining the methylation states or the binding states of the one or more target regions.

In some embodiments, method 200 may include or may exclude one or more additional operations (operation 222). For example, method 200 may exclude amplification of the one or more target regions.

In some embodiments of method 200, there may be additional or fewer operations. Furthermore, the order of the operations may be changed, and/or two or more operations may be combined into a single operation.

We now further describe embodiments of the analysis techniques. These analysis techniques may provide additional techniques for analysis of methylation of targeted portions or regions of molecules of DNA.

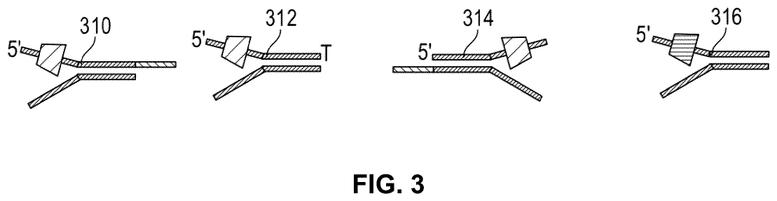

FIG. 3 presents a drawing illustrating an example of motor-protein adapter options. The options for motor protein adapters include: a motor adapter with native barcoding 310, a motor adapter without native barcoding 312, a motor protein for RNA sequencing 314 (such as a dRNA adapter), and/or a complementary DNA (cDNA) 316 adapter. Note that the adapter arrangement may include an dRNA kit adapter with the motor protein on 3′ end of a molecule in the DNA.

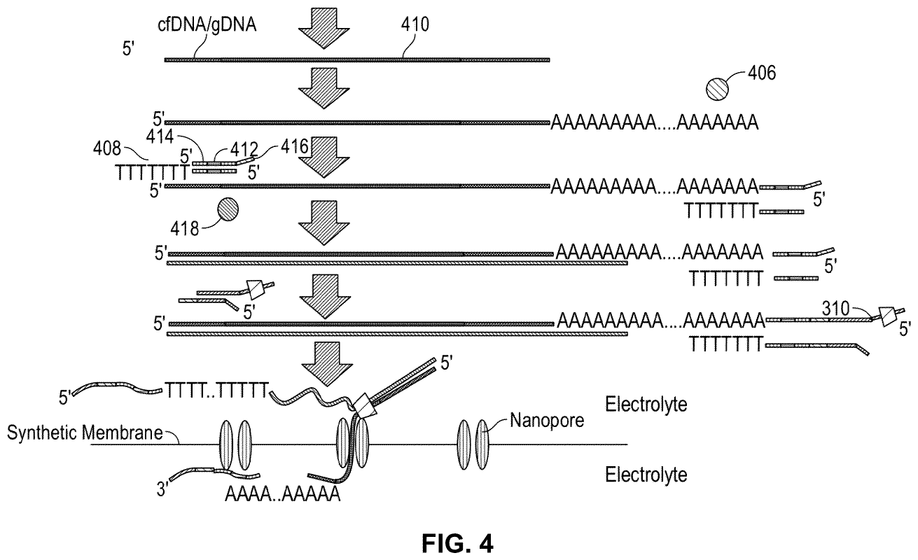

FIG. 4 presents a drawing illustrating an example of a technique for processing a sample with single-strand DNA. This processing may include a Terminal Deoxynucleotidyl Transferase (TdT)-mediated ligation and library preparation (including sequential anneal and adapter ligation operations. Notably, oligo-based hybrid capture may be performed, resulting in elution of single-strand molecules of DNA from target regions of interest 410 in cell-free DNA (cfDNA) or germline DNA (gDNA). In some embodiments, end-repair may be performed prior to hybrid capture to fix internal nicks and generate 3′OH for subsequent ligation. Then, perform terminal transferase 406 non-templated 3′ extension of a homopolymer. Note that the tail length distribution can be controlled by altering a ratio of 3′ DNA ends and Deoxynucleotide Triphosphates (dNTPs). The tail may be N6-Methyladenosine (m6A) in order to be able to distinguish from Adenine-fragment ends.

A library preparation (LP) adapter 408 may be bonded to the sample. LP adapter 408 may include: a 3′ poly Thymidine tail, on the double-stranded DNA end, a sample index 412, constant flanking regions 414, and a 3′ overhang tail 416 on the single-stranded DNA end. Note that we may have to use barcoded adapter, and the adapter may have a DNA-optimized motor protein attached on 3′ end.

In some embodiments, optional complementary strand synthesis may be performed if necessary for improved sequencing throughput. This operation may use DNA polymerase 418 with an intrinsic 3′→5′ exonuclease activity.

Next, motor protein adapter ligation 420 may be performed using a sticky end y-adapter with a bound motor protein.

The resulting sample may enable membrane analysis (e.g., using a nanopore or a synthetic membrane immersed in an electrolyte). Note that this analysis technique may be used without targeting for whole-genome sequencing, because it may capture single-strand DNA (as opposed to only capturing double-strand DNA). The addition of a homopolymer trail may help short fragments survive cleanup operations by making them longer.

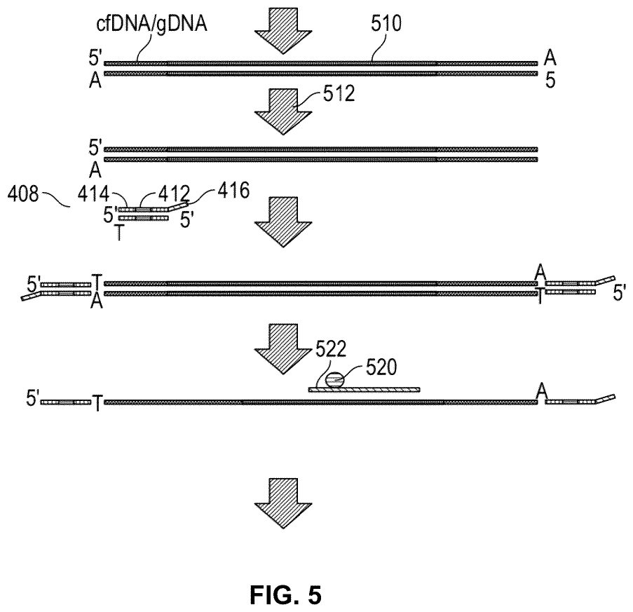

FIG. 5 presents a drawing illustrating an example of a technique for processing a sample. Notably, FIG. 5 presents a two-ligation operation technique with additional A-tailing (or a non-template nucleotide enzymatically added to the 3′ end of a blunt, double-strand molecule of DNA). Notably, library preparation, end-repair and dA tailing may be performed about a target region 510 with a remainder of the modules of DNA being cell-free DNA or germline DNA. Then, a phosphate treatment 512 removes a 5′ P group from the molecules of DNA to prevent 5′ ligation. Moreover, the sample may be ligated with a ‘sample index adapter’ using T4 DNA ligase. This may add a sample index and generate a substrate for subsequent ligation to a membrane or LP adapter 408. LP adapter 408 may include a sample index 412 and flanking constant regions 414 for universal hybridization, and an overhand tail 416 for subsequent use with the analysis membrane. In some embodiments, the ligation-strand of adapter may include sequence base(s) at 3′ end that form an overhand for the subsequent ligation operation (e.g., A). Note that LP adapter 408 may include: a 3′ Thymidine tail on the double-stranded DNA end, and a 3′ overhang tail 416 on the single-stranded DNA end. Next, target enrichment may be performed with biotinylated nucleic acid probes 522 through binding, via streptavidin-magnetic beads, to a biotin-DNA complex 520. In some embodiments, greater than or equal to 80 nucleotide probes may be used for specific enrichment.

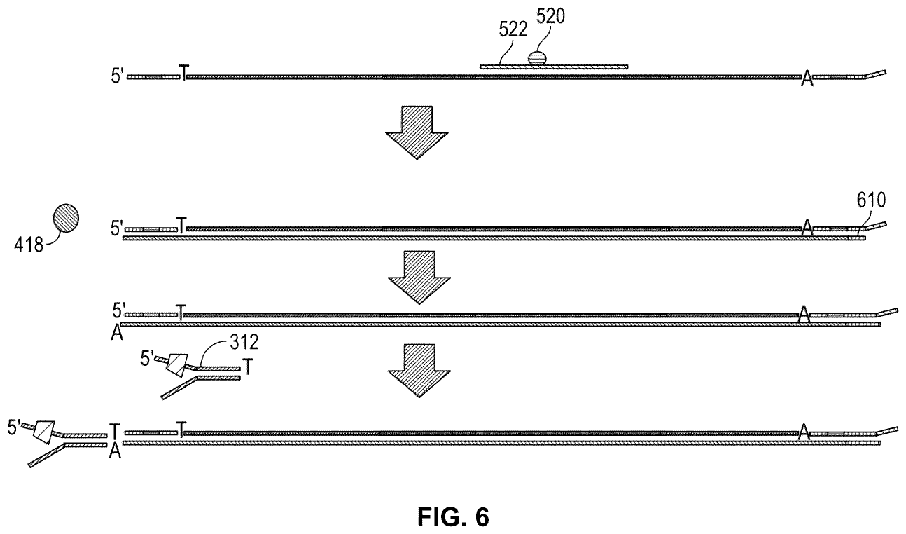

FIG. 6 presents a drawing illustrating an example of a technique for processing a sample using a two-ligation operation technique with additional A-tailing. Notably, the elution of target DNA from streptavidin beads and hybridization to a universal primer sequence 610 may create a double-strand DNA end with 3′ overhang/sticky end tail (greater than 1 base). In some embodiments, the extension with DNA polymerase 418 with an intrinsic 3′→5′ exonuclease activity may create a double-strand DNA library with 3′ overhang/sticky end on the original DNA strand. This sticky end may not be A tailed. Note that the universal primer hybridization and DNA polymerase extension operation may optionally be performed ‘on bead’ with a strand displacing polymerase to elute library from a streptavidin magnetic bead complex in a single operation. Next, directional ligation of a motor adapter may be performed with T4 DNA ligase (Thymidine-tailed with a bound motor protein).

Note that the strand that has the motor protein attached may be the one that gets sequenced. However, if in any strategy the newly synthesized complementary strand also gets sequenced, modified dNTPs may be added (such as m6A, 5-Hydroxymethylcytosine or 5hmC, 5-Methylcytosine or 5mC, etc.) and base analogs (such as Bromodeoxyuridine or BrdU and, more generally, techniques for identifying end repair bases) may be used to identify the newly synthesized strand that will not harbor endogenous methylation. While cytosine-5)-methyltransferase 1 (DNMT1) enzyme efficiency may not be sufficient, another option is to copy endogenous DNA methylation onto the newly synthesized strand using DNMT1, which may preferentially be methylated at hemimethylated sites.

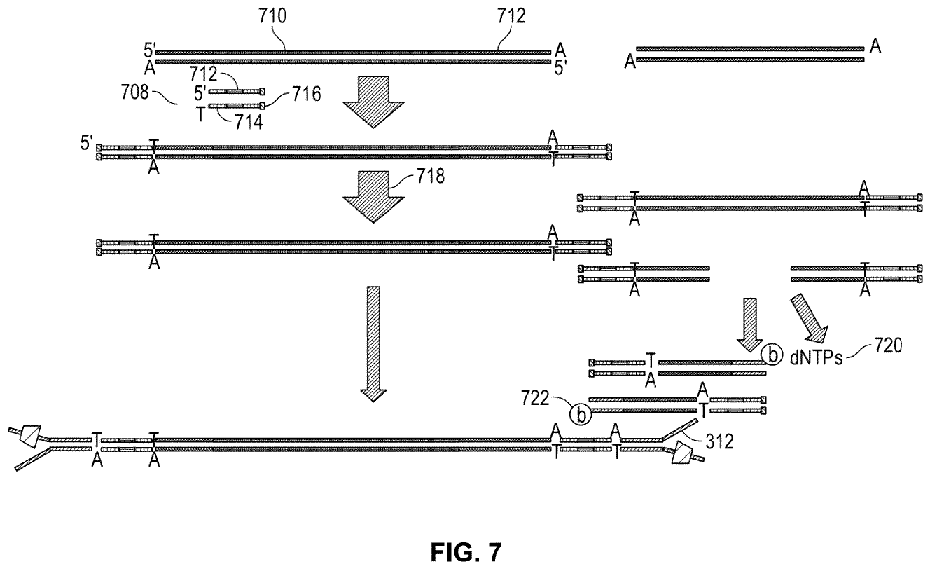

FIG. 7 presents a drawing illustrating an example of a technique for processing a sample by depleting off-target regions in molecules of DNA. Notably, the depletion or removal of off-target regions 710 relative to target regions 712 may use Cas9 or a restriction enzyme. In some embodiments, methylation-sensitive/dependent restriction enzymes may be used to similarly deplete the sample. Note that this may not just be based at least in part on the sequence, but may also be based at least in part on the methylation status.

Then, LP adapter 708 may be attached to the sample. LP adapter 708 may include: a sample index 712 and flanking constant regions 714, and a protected end 716. Note that LP adapter 708 may include: a 3′ Thymidine tail on the double-stranded DNA end.

Moreover, the sample may be digested 718 with restriction enzymes or Cas9 with a pool of guide RNA (gRNA) targeting regions outside the target regions of interest 712.

Note that exonuclease treatment 720 (e.g., using dNTPs) may be used to remove off-target sites that have exposed ends revealed by Cas9 or restriction enzyme digestion. Alternatively or additionally, unblocked ends in off-target regions may be ligated to biotinylated adapters 722 (such as streptavidin magnetic beads) for pulldown and removal from the pool.

A given region of interest may have its ends de-blocked. A-tailing and ligation of Thymidine-tailed motor protein adapter 312 may result in a fragment suitable for sequencing. Note that both strands may be sequenced because the sample is double stranded and both have an attached motor protein.

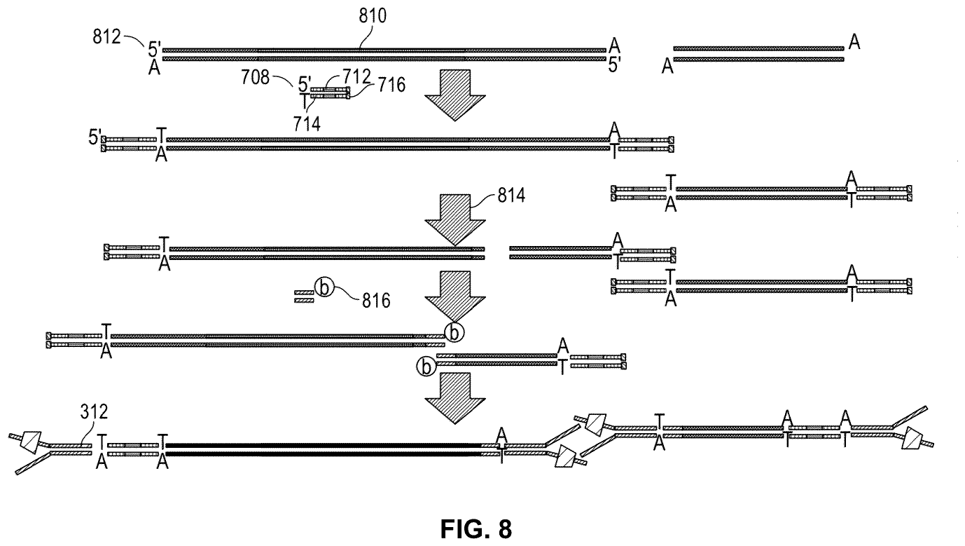

FIG. 8 presents a drawing illustrating an example of a technique for processing a sample by enriching the sample using a Cas9 protein or a restriction enzyme. Notably, this approach is similar to reduced representation bisulfite sequencing (RRBS) or Cas9-based ligation, but may only require cutting one end of the molecule. In some embodiments, a methylation-sensitive/dependent restriction enzymes may be used to enrich, not just based at least in part on the sequence, but also based at least in part on the methylation status. When a region of interest (such as target region 810) has a target motif that is too densely populated (which may result in fragments that are too short for successful sequencing), a blocker like dCas9 or a dead restriction enzyme may be used to better preserve those regions by binding on/adjacent to cut sites to protect them.

After end repair and bonding of A-tailed DNA 812, LP adapter 708 may be attached to the sample. LP adapter 708 may include: a sample index 712 and flanking constant regions 714, and a protected end 716. Note that LP adapter 708 may include: a 3′ Thymidine tail on the double-stranded DNA end.

Then, the sample may be digested 814 with restriction enzymes or Cas9 with pool of gRNAs targeting regions in/near of region of interest. In some embodiments, the sample may be barcoded in this biotinylated adapter to identify sample for fragments cut more than once.

Moreover, the sample may be ligated and isolated using streptavidin magnetic beads 816.

Next, the biotin may be removed by having an adapter sequence with a rare restriction enzyme motif or a methylated adenine (mA)-dependent restriction enzyme motif (e.g., the restriction enzyme DpnI recognizes GmATC). Alternatively, photocleavable biotin may be used.

Furthermore, the ends of the sample may be de-blocked, A-tailed and T-tailed motor adapter 312 may be ligated onto the sample to produce fragments for sequencing. Note that both strands can be sequenced because the sample is double stranded and both strands have a motor protein attached.

If multiple cut sites occur within a single molecule, fragments that do not have end barcoded adapters and are internal may not be barcoded and may still get the motor protein ligated because this would be repaired during A-tailing.

In some embodiments, a rare/mA-dependent restriction enzyme may digest the site in the edge of the barcoded adapters and may leave a sticky overhang for the sticky version of the motor protein adapter. Then, there may only be a motor protein adapter on one end of the fragment, but it would be barcoded.

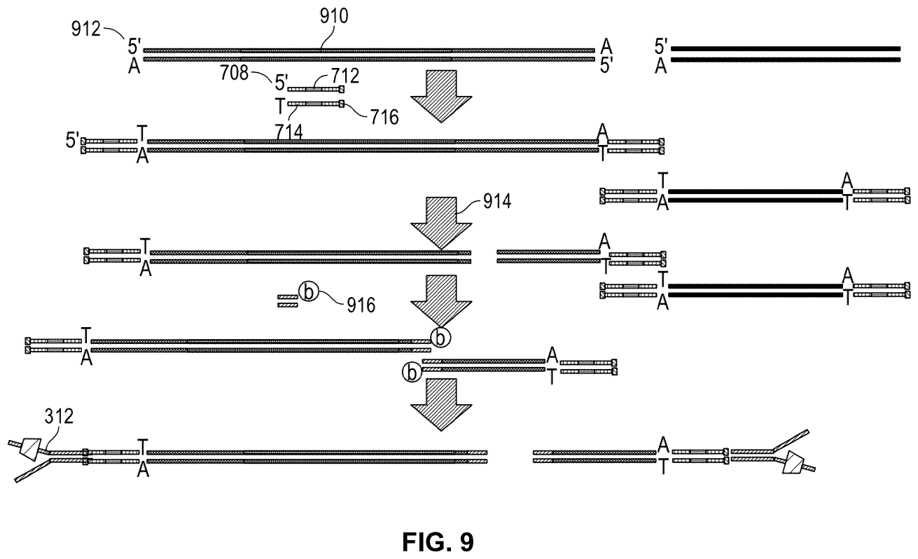

FIG. 9 presents a drawing illustrating an example of a technique for processing a sample by enriching the sample using a Cas9 protein or a restriction enzyme. Notably, this approach is similar to RRBS or Cas9-based ligation, but may only require cutting one end of the molecule. In some embodiments, a methylation-sensitive/dependent restriction enzymes may be used to enrich, not just based at least in part on the sequence, but also based at least in part on the methylation status. When a region of interest (such as target region 910) has a target motif that is too densely populated (which may result in fragments that are too short for successful sequencing), a blocker like dCas9 or a dead restriction enzyme may be used to better preserve those regions by binding on/adjacent to cut sites to protect them.

After end repair and bonding with A-tailed DNA 912, LP adapter 708 may be attached to the sample. LP adapter 708 may include: a sample index 712 and flanking constant regions 714, and a protected end 716. Note that LP adapter 708 may include: a 3′ Thymidine tail on the double-stranded DNA end.

Then, the sample may be digested 914 with restriction enzymes or Cas9 with pool of gRNAs targeting regions in/near of region of interest. In some embodiments, this biotinylated adapter may be barcoded to identify the sample for fragments that are cut more than once.

Moreover, the sample may be ligated and isolated using streptavidin magnetic beads 916.

Next, the biotin may be removed by having an adapter sequence with a rare restriction enzyme motif or a mA-dependent restriction enzyme motif (e.g., the restriction enzyme DpnI recognizes GmATC). Alternatively, photocleavable biotin may be used.

Furthermore, the ends of the sample may be de-blocked.

Additionally, restriction enzymes may digest barcoded adapters to reveal sticky ends complementary to a sticky-end motor protein adapter, so that only fragments that have barcodes are sequenced (otherwise, we could suffer from internally cut fragments without barcodes being sequenced). Alternatively, there may be a sample barcode in a biotinylated adapter and the same sticky end may be exposed within the biotinylated adapter for final ligation at both ends.

Note that if multiple cut sites occur within a single molecule, fragments that do not have end barcoded adapters and are internal may not be barcoded and may still get the motor protein ligated because this would be repaired during A-tailing.

In some embodiments, a rare/mA-dependent restriction enzyme may digest the site in the edge of the barcoded adapters and may leave a sticky overhang for the sticky version of the motor protein adapter. Then, there may only be a motor protein adapter on one end of the fragment, but it would be barcoded.

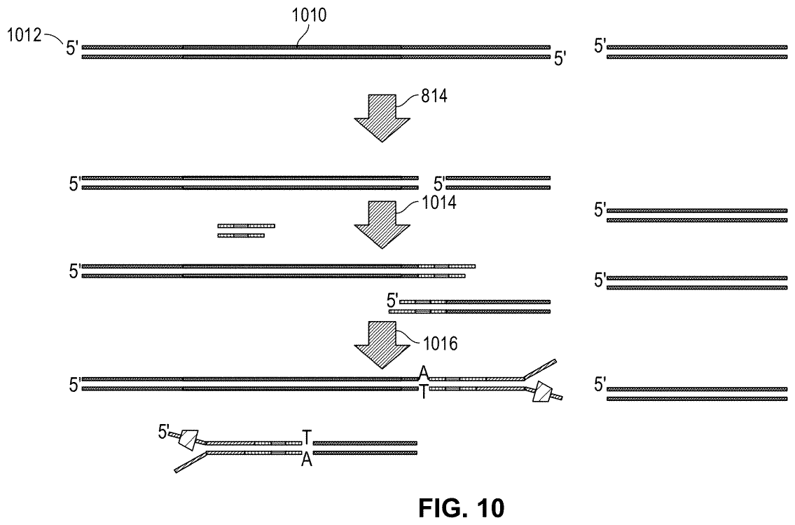

FIG. 10 presents a drawing illustrating an example of a technique for processing a sample by enriching the sample using a Cas9 protein or a restriction enzyme. Notably, this approach is similar to RRBS or Cas9-based ligation, but may only require cutting one end of the molecule. In some embodiments, a methylation-sensitive/dependent restriction enzymes may be used to enrich, not just based at least in part on the sequence, but also based at least in part on the methylation status. When a region of interest (such as target region 1010) has a target motif that is too densely populated (which may result in fragments that are too short for successful sequencing), a blocker like dCas9 or a dead restriction enzyme may be used to better preserve those regions by binding on/adjacent to cut sites to protect them.

After end repair and bonding with A-tailed DNA 1012, blunt ligation 1014 may be performed to newly cut ends or whatever ends the restriction enzyme leaves. Furthermore, ligation with a motor protein adapter 1016 may be performed with a restriction that reveals complementary sticky ends.

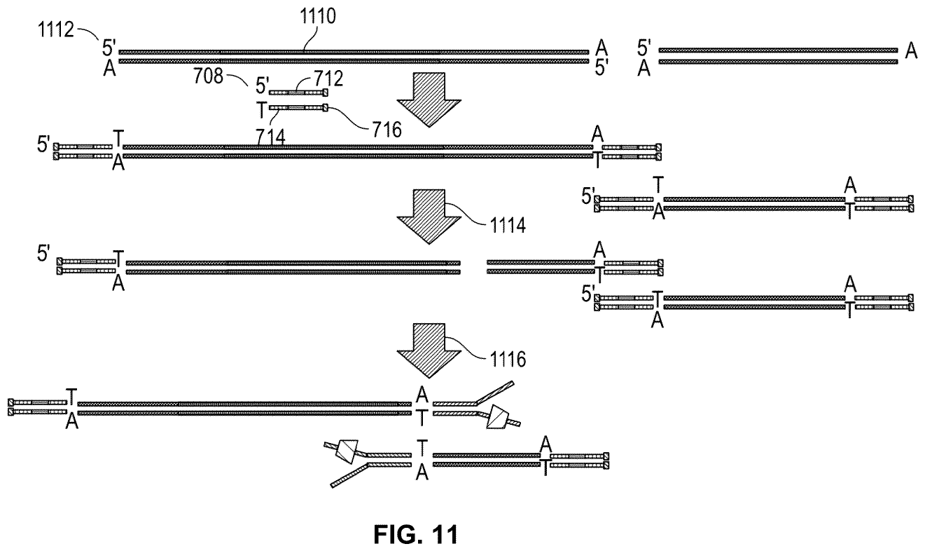

FIG. 11 presents a drawing illustrating an example of a technique for processing a sample by enriching the sample using a Cas9 protein or a restriction enzyme. Notably, this approach is similar to RRBS or Cas9-based ligation, but may only require cutting one end of the molecule. In some embodiments, a methylation-sensitive/dependent restriction enzymes may be used to enrich, not just based at least in part on the sequence, but also based at least in part on the methylation status. When a region of interest (such as target region 1110) has a target motif that is too densely populated (which may result in fragments that are too short for successful sequencing), a blocker like dCas9 or a dead restriction enzyme may be used to better preserve those regions by binding on/adjacent to cut sites to protect them.

After end repair and bonding with A-tailed DNA 1112, LP adapter 708 may be attached to the sample. LP adapter 708 may include: a sample index 712 and flanking constant regions 714, and a protected end 716. Note that LP adapter 708 may include: a 3′ Thymidine tail on the double-stranded DNA end.

Then, the sample may be digested 1114 with restriction enzymes or Cas9 with pool of gRNAs targeting regions in/near of region of interest.

Moreover, the final adapter may be ligated with a motor protein 1116 (e.g., A-tail or blunt). Next, only one of the two strands per molecule may be sequenced.

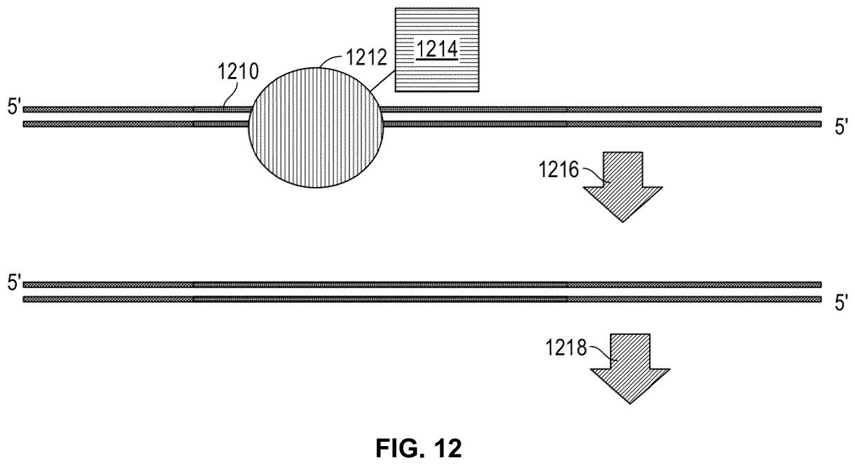

FIG. 12 presents a drawing illustrating an example of a technique for processing a sample using dRE enrichment of target regions 1210. Notably, an isolated DNA sample may be bound to dRE 1212. For example, dRE 1212 may be the biding domain of: a restriction endonuclease (such as Mspl), a restriction endonuclease (such as BsiSI), a methylation-sensitive restriction enzyme (such as Hpall), and/or a restriction endonuclease (such as Hapll for the CCGG motif) to enrich for CGI. Note that dRE 1212 may include a tag 1214, such as an anti-V5 tag antibody (Biotin). Then, the dRE-bound DNA may be pulled down 1216, and the resulting sample may be analyzed 1218 using an analysis membrane (such as a nanopore) without further modification.

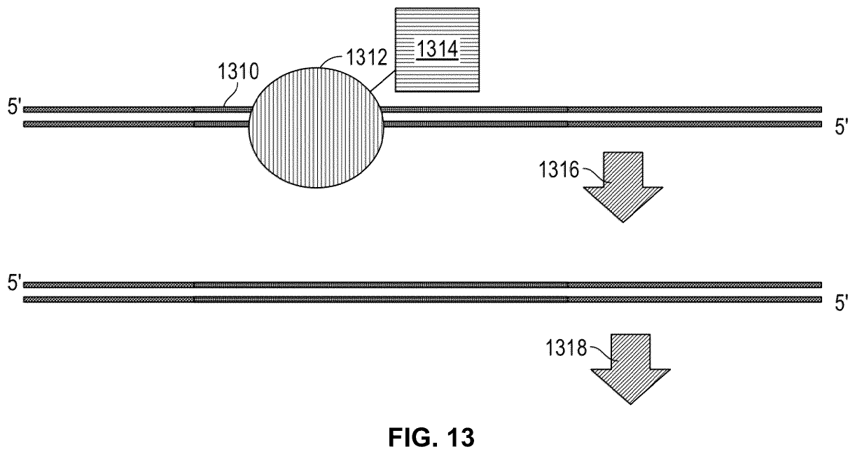

FIG. 13 presents a drawing illustrating an example of a technique for processing a sample using MBD protein 1312 enrichment of target regions 1310. Notably, an isolated cell-free or germline DNA sample may be bound to MBD protein 1312. For example, this approach can be used to enrich for methylation with MBD protein 1312 that prefers a methyl-CpG-binding domain (mCpG). Alternatively or additionally, MBD protein 1312 may bind to CpG and 5 mCpG (double-strand DNA dyads) with similar affinity, so it may enrich for CpG-enriched regions (CGI). Note that MBD protein 1312 may include a tag 1314, such as an anti-V5 tag antibody (Biotin). Then, the MBD-bound DNA may be pulled down 1316, and the resulting sample may be analyzed 1318 using an analysis membrane (such as a nanopore) without further modification.

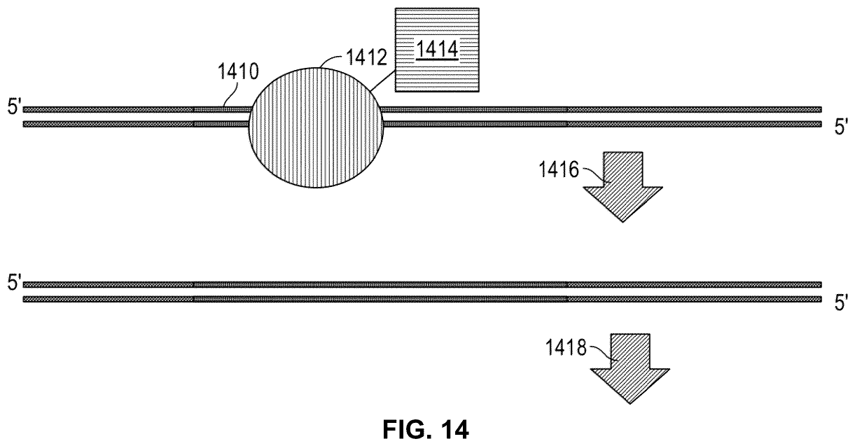

FIG. 14 presents a drawing illustrating an example of a technique for processing a sample using dCas9 enrichment of target regions 1410. Notably, an isolated cell-free or germline DNA sample may be bound to dCas9 1412. Note that dCas9 1412 may include a tag 1414, such as an anti-V5 tag antibody (Biotin). Then, the dCas9-bound DNA may be pulled down 1416, and the resulting sample may be analyzed 1418 using an analysis membrane (such as a nanopore) without further modification.

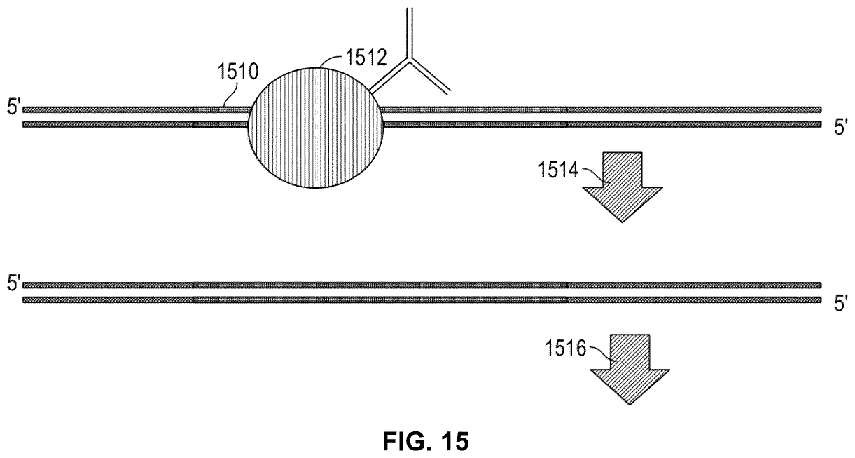

FIG. 15 presents a drawing illustrating an example of a technique for processing a sample using chromatin immunoprecipitation (ChIP) enrichment of target regions 1510. Notably, an isolated cell-free or germline DNA sample may be bound to a Histone/key transcription factor 1512. This may provide a plasma chromatic-like technique for pulling or extracting out protein-bound DNA. For example, Trimethylation of Histone H3 at Lysine 4 or H3K4me3 (which may mark active and poised promoters) may be marked with ChIP and the sequence may be pulled down 1514 for analysis using an analysis membrane or nanopore. Thus, the resulting sample may be analyzed 1516 using an analysis membrane (such as a nanopore) without further modification.

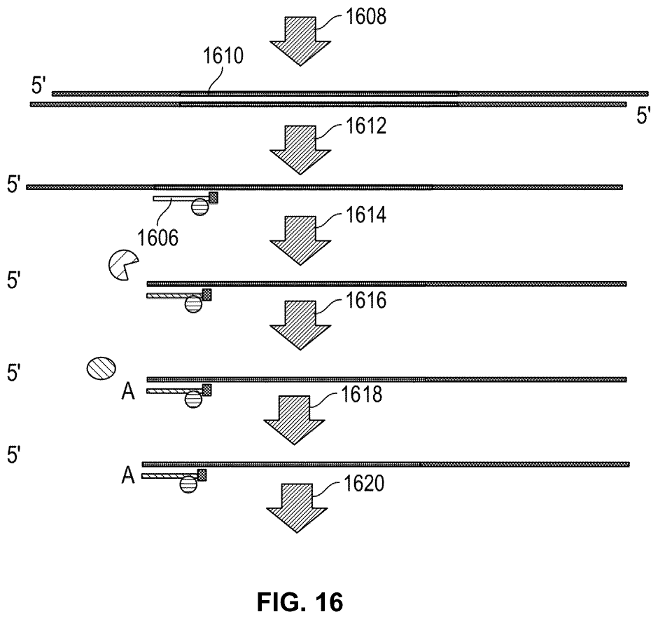

We now describe modified probe capture techniques for an adapter with a motor protein on 5′ end of molecules in a DNA sample. FIG. 16 presents a drawing illustrating an example of a technique for processing a sample (with target regions 1610) using a probe capture technique for a 5′ motor protein adapter. Notably, in a cell-free DNA or germline DNA sample, optionally perform end-repair 1608 prior to denaturation to fix internal nicks that could reduce sequencing yield. A ligation operation may be needed. For example, at least one modified dNTP may be used to mark filled-in region in sequencing, which may improve methylation/somatic calling accuracy.

Then, perform denature and biotin-probe 1612 hybridization. For example, target one stand of DNA, both strands of DNA, tile the probes (overlapping and/or non-overlapping), etc. Note that biotin probe 1612 may include a 5′ primer ligation block 1606, e.g., a 5′ OH group.

Moreover, a 5′→3′ exonuclease treatment 1614 may be performed. Next, an A-tailing treatment 1616 may be performed. In some embodiments, optionally perform blunt adapter ligation.

The reaction may then be cleaned up to remove the exonuclease and a mesophilic DNA polymerase possessing A-tailing activity may be added with Adenosine Triphosphate (ATP), and may be incubated at a moderate temperature (e.g., approximately 37 C). Note that A-tailing may be skipped if blunt ligation may be performed. Moreover, adapter dimers may be well-tolerated in the workflow by adding a magnetic streptavidin bead-based cleanup operation after ligation that will remove adapter dimers.

Next, target enrichment 1618 may be performed through streptavidin-magnetic beads binding to biotin-DNA complex. In some embodiments, the order of the enrichment operation and the previous two operations may be swapped. This may be advantageous depending on stability of A-tail in hybridization reaction (if there is exonuclease contamination).

Furthermore, membrane adapter (such as a nanopore adapter) ligation 1620 may be performed. There may be several variations on this operation. For example, the bound primer on the DNA may be extended and the temperature may be elevated before enrichment to increase enrichment stringency (OTR). Alternatively or additionally, there may not be an enrichment operation. Instead, primer binding may be used selectively for on-targets. Only these targets may be prepped with membrane adapters.

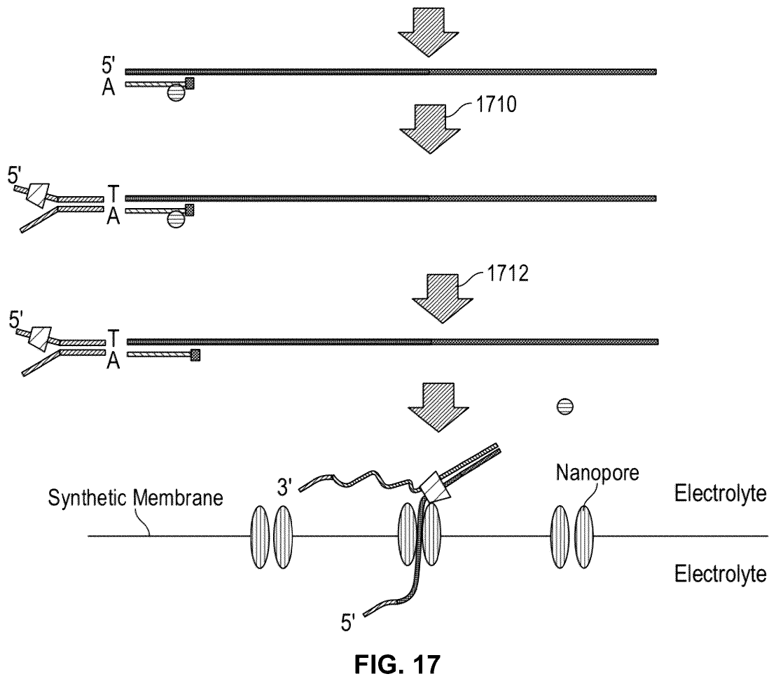

FIG. 17 presents a drawing illustrating an example of a technique for processing a sample using a probe capture technique for an adapter. Notably, the membrane-adapter ligation 1710 may include a Thymidine-tailed y-adapter with a bound motor protein. In some embodiments, there may be an optional cleanup ligation 1712 product, which may remove enzymes, buffers and adapter dimers that were produced (e.g., they may have been enabled through streptavidin magnetic beads). Alternatively or additionally, other options may include: photoclavable biotin, a probe without biotin and/or only using ligation where the primer is bound for double-stranded ligation.

Variations on these operations may include: extending the bound primer on the DNA and elevating the temperature before enrichment to increase enrichment stringency (OTR). Alternatively or additionally, there may not be an enrichment operation. Instead, primer binding may be used selectively for on-targets. Only these targets may be prepped with membrane adapters.

Note that attachment of an analysis membrane ‘tether’ by annealing to the bottom adapter oligo prior to sequencing is not shown in FIG. 17. Some workflows may not use the ‘tether’ for sequencing. Thus, this operation may be considered optional in all of the disclosed analysis techniques.

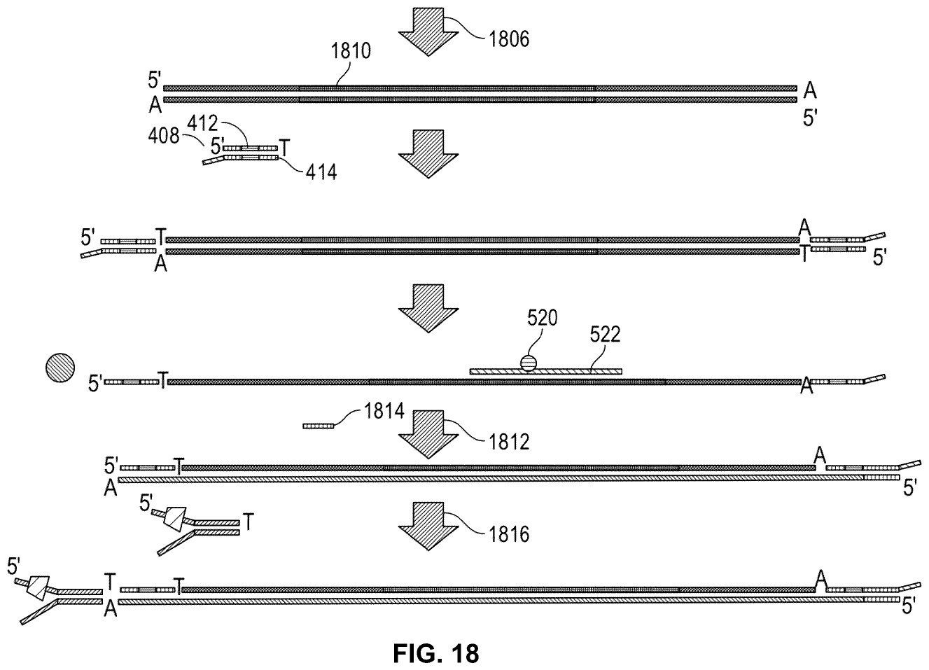

FIG. 18 presents a drawing illustrating an example of a two-ligation technique for processing a sample. Notably, library preparation, end-repair and dA tailing 1806 may be performed as first operations in the analysis technique about a target region 1810 with a remainder of the modules of DNA being cell-free DNA or germline DNA. Then, the sample may be ligated with a ‘sample index adapter’ using T4 DNA ligase. This may add a sample index and generate a substrate for subsequent ligation to a membrane or LP adapter 408. LP adapter 408 may include a sample index 412 and flanking constant regions 414 for universal hybridization. Moreover, LP adapter 408 may include: a 3′ Thymidine tail on the double-stranded DNA end. Next, target enrichment may be performed with biotinylated nucleic acid probes 522 through binding, via streptavidin-magnetic beads, to a biotin-DNA complex 520. In some embodiments, greater than or equal to 80 nucleotide probes may be used for specific enrichment.

Moreover, elution 1812 of target DNA may be performed from streptavidin beads and hybridization to a universal primer sequence 1814. Furthermore, the sample may be extended with DNA polymerase with an intrinsic 3′→5′ exonuclease activity to create a double-strand DNA library with an A tail for subsequent adapter ligation. Note that the universal primer hybridization and DNA polymerase extension operation may be optionally performed ‘on bead’ with strand displacing polymerase to elute a library from the streptavidin magnetic bead complex in a single operation. Furthermore, directional ligation 1816 of an analysis membrane adapter may be performed with T4 DNA ligase (such as Thymidine-tailed with a bound motor protein).

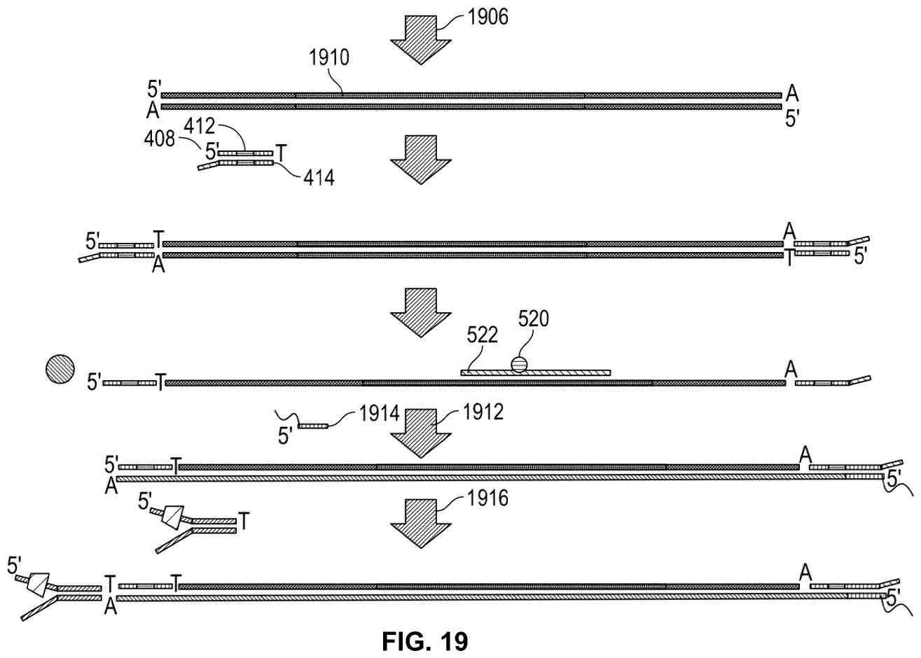

FIG. 19 presents a drawing illustrating an example of a two-ligation technique for processing a sample. This two-ligation technique may include a universal primer with a 5′ hydrophobic group (or ‘tether’) to improve sequencing yield. Note that this modification may be applied to other disclosed embodiments by modifying the universal primer or adapter at the 5′ end.

Notably, library preparation, end-repair and dA tailing 1906 may be performed as first operations in the analysis technique about a target region 1910 with a remainder of the modules of DNA being cell-free DNA or germline DNA. Then, the sample may be ligated with a ‘sample index adapter’ using T4 DNA ligase. This may add a sample index and generate a substrate for subsequent ligation to a membrane or LP adapter 408. LP adapter 1908 may include a sample index 412 and flanking constant regions 414 for universal hybridization. Moreover, LP adapter 408 may include: a 3′ Thymidine tail on the double-stranded DNA end. Next, target enrichment may be performed with biotinylated nucleic acid probes 522 through binding, via streptavidin-magnetic beads, to a biotin-DNA complex 520. In some embodiments, greater than or equal to 80 nucleotide probes may be used for specific enrichment.

Furthermore, elution 1912 of target DNA may be performed from streptavidin beads and hybridization to universal primer sequence. Additionally, the sample may be extended with DNA polymerase 1914 with an intrinsic 3′→5′ exonuclease activity to create a double-strand DNA library with an A tail for subsequent adapter ligation. Note that the universal primer hybridization and DNA polymerase extension operation may be optionally performed ‘on bead’ with strand displacing polymerase to elute a library from the streptavidin magnetic bead complex in a single operation. In some embodiments, the universal primer sequence may include a 5′ lipid/hydrophobic group. Furthermore, directional ligation 1916 of an analysis membrane adapter may be performed with T4 DNA ligase (such as Thymidine-tailed with a bound motor protein).

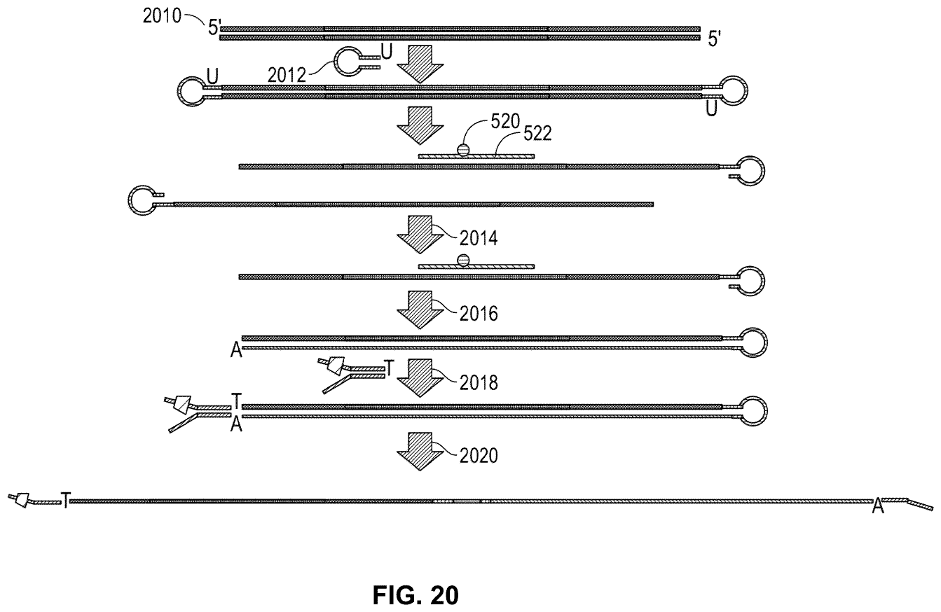

FIG. 20 presents a drawing illustrating an example of a technique for processing a sample using a dumbbell-shaped adapter. This approach may be similar to the two-ligation technique, but the adapter may serve as the primer. In this approach, the sample may be optionally A-tailed 2010. Then, dumbbell ligation may be performed. Adapter 2012 may contain a cell barcode and Uracil for cleavage. Moreover, the sample may be enriched using biotinylated nucleic acid probes 522 through binding, via streptavidin-magnetic beads, to a biotin-DNA complex 520. Next, pulldown 2014 may be performed. Furthermore, the sample may be extended with DNA polymerase 2016 with an intrinsic 3′→5′ exonuclease activity to create a double-strand DNA library with an A tail for subsequent adapter ligation 2018. Additionally, following motor protein ligation 2018, the sample may become single-strand DNA 2020 as sequenced.

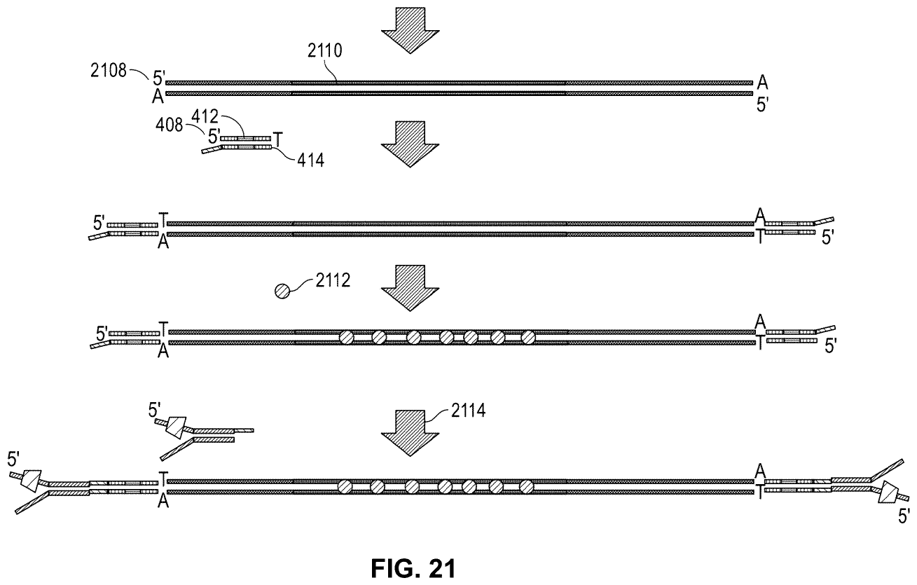

We now further describe the analysis technique illustrated previously in FIG. 13. Notably, FIG. 21 presents a drawing illustrating an example of a technique for processing a sample using a hybrid MBD protein. Library preparation, end-repair and dA tailing 2108 may be performed as first operations in the analysis technique about a target region 2110 with a remainder of the modules of DNA being cell-free DNA or germline DNA.

Then, the sample may be ligated with a ‘sample index adapter’ using T4 DNA ligase. This may add a sample index and generate a substrate for subsequent ligation to a membrane or LP adapter 408. LP adapter 408 may include a sample index 412 and flanking constant regions 414 for universal hybridization. Moreover, LP adapter 408 may include: a 3′ Thymidine tail on the double-stranded DNA end.

Moreover, one or more locations in target region 2110 may be bound to MBD proteins 2112. Furthermore, motor adapters may be bound 2114 to the MBD-bound DNA.

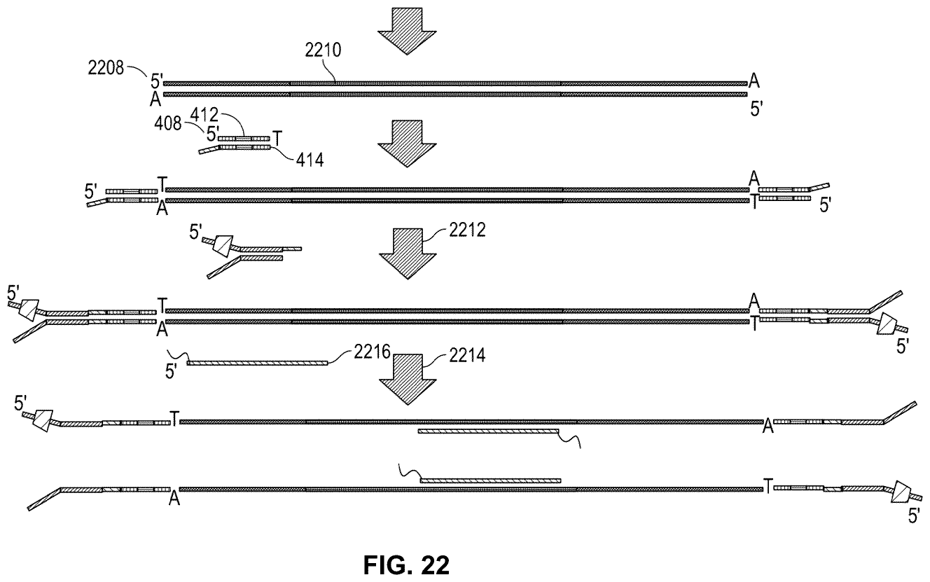

We now describe in-flow enrichment with tether enrichment probes. FIG. 22 presents a drawing illustrating an example of a two-ligation technique for processing a sample using tether enrichment probes. Notably, library preparation, end-repair and dA tailing 2208 may be performed as first operations in the analysis technique about a target region 2210 with a remainder of the modules of DNA being cell-free DNA or germline DNA. Then, the sample may be ligated with a ‘sample index adapter’ using T4 DNA ligase. This may add a sample index and generate a substrate for subsequent ligation to a membrane or LP adapter 408. LP adapter 2208 may include a sample index 412 and flanking constant regions 414 for universal hybridization. Moreover, LP adapter 408 may include: a 3′ Thymidine tail on the double-stranded DNA end.

Next, perform directional ligation 2212 of analysis membrane adapters with T4 DNA ligase. Note that analysis membrane adapters may include bound motor protein. Furthermore, target region 2210 may be bound with probes 2214 with a 5′ lipid/hydrophobic group ‘tether’ 2216.

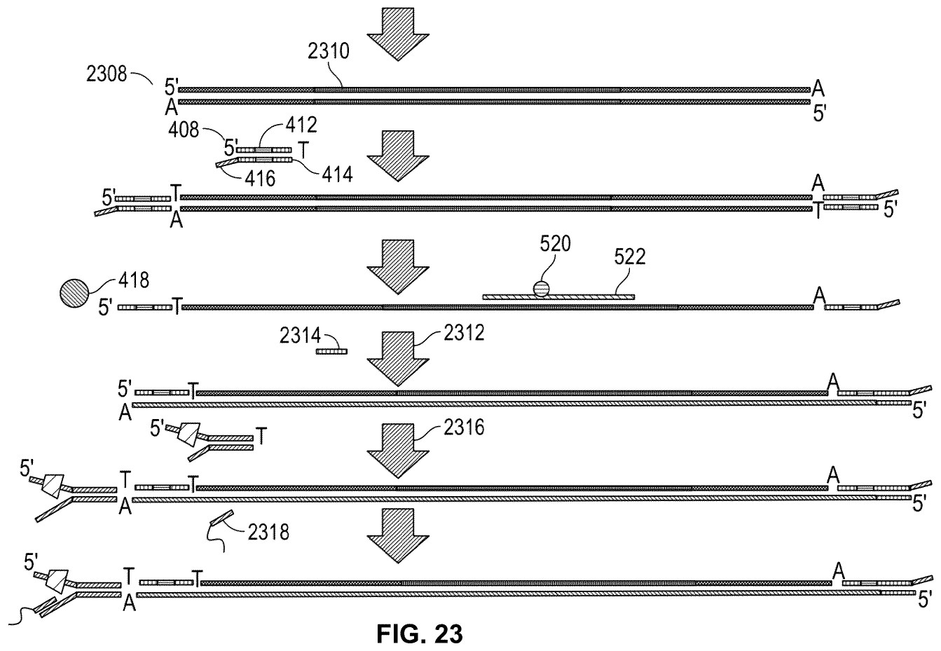

We now describe an example of a two-ligation technique with inter-library annealing for increased sequencing yield. FIG. 23 presents a drawing illustrating an example of a two-ligation technique for processing a sample. Notably, library preparation, end-repair and dA tailing 2308 may be performed as first operations in the analysis technique about a target region 2310 with a remainder of the modules of DNA being cell-free DNA or germline DNA. Then, the sample may be ligated with a ‘sample index adapter’ using T4 DNA ligase. This may add a sample index and generate a substrate for subsequent ligation to a membrane or LP adapter 408. LP adapter 408 may include a sample index 412 and flanking constant regions 414 for universal hybridization. Moreover, LP adapter 408 may include: a 3′ Thymidine tail on the double-stranded DNA end a 3′ overhand analysis adapter hybridization sequence 416. This 3′ overhand analysis adapter hybridization sequence (which may have partial or full sequence overlap to a tether oligonucleotide) may improve direction ligation efficiency and may improve subsequent sequencing.

Next, target enrichment may be performed with biotinylated nucleic acid probes 522 through binding, via streptavidin-magnetic beads, to a biotin-DNA complex 520. In some embodiments, greater than or equal to 80 nucleotide probes may be used for specific enrichment.

Moreover, elution 2312 of target DNA may be performed from streptavidin beads and hybridization to universal primer sequence 2314. Furthermore, the sample may be extended with DNA polymerase 418 with an intrinsic 3′→5′ exonuclease activity to create a double-strand DNA library with an A tail for subsequent adapter ligation. Note that the universal primer hybridization and DNA polymerase extension operation may be optionally performed ‘on bead’ with strand displacing polymerase to elute a library from the streptavidin magnetic bead complex in a single operation. Furthermore, directional ligation 2316 of an analysis membrane adapter may be performed with T4 DNA ligase (such as Thymidine-tailed with a bound motor protein). Because of the complementarity in the membrane adapter with a membrane adapter hybridization sequence, some inter- and intra-molecular annealing may be possible. This may be transient and out completed by excess tether oligonucleotide added in the subsequent operation.

Furthermore, a tether oligonucleotide 2318 (with lipid 5′ end) may be annealed to the 3′ end of the analysis membrane adapter. Tether 2318 may increase sequencing yield/throughput by being attracted/pulled down to the analysis membrane during sequencing. Note that the tether oligonucleotide 2318 may be pre-annealed to the analysis membrane adapter prior to the previous operation of ligation to disfavor inter- and intra-molecular annealing of library molecules.

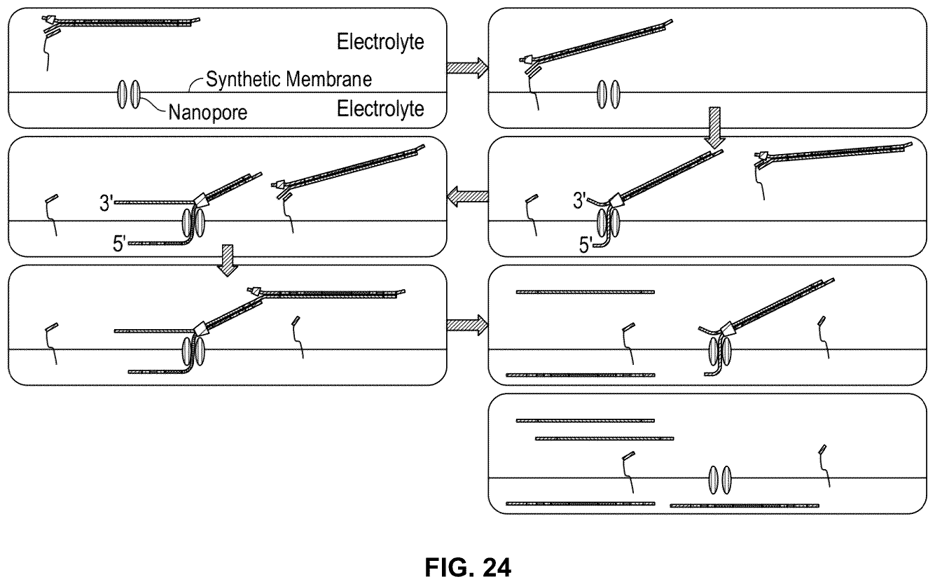

FIG. 24 presents a drawing illustrating an example of a technique for processing a sample with surface-localized annealing. Note that the tether oligonucleotide may have a sequence similarity to the ‘adapter hybridization sequence’ on the adapter distal to the pore protein. Moreover, the tether oligonucleotide may localize a first library molecule to the surface, near a pore occupied by a second library molecule. The tether oligonucleotide may be displaced from a first library molecule to allow the first library molecule to anneal to second library molecule, thereby bringing it near to the pore opening. Once the second library molecule is sequenced, the adapter protein of the first library molecule may immediately occupy the pore.

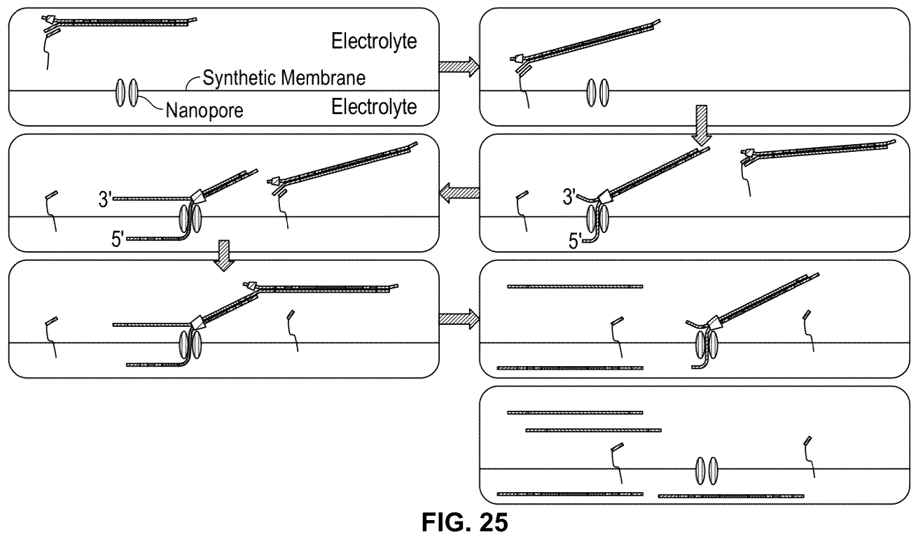

FIG. 25 presents a drawing illustrating an example of a technique for processing a sample with surface-localized annealing. Note that two or more library molecules may anneal to each other as well during ligation. This may not necessarily be bad, because it may be as if the library molecules are brought down to surface together, and may be processed sequentially through the pore with low dwell time. Thus, this may achieve one of the purposes of the analysis techniques. However, longer concatemers (which may not be non-covalently connected) of molecules may diffuse slower to the surface. Consequently, the leading library molecule in the concatemer may still be bound to a tether.

Alternatively or additionally, there may be low-level transient annealing between library ends as they are in low concentration. Each individual library molecule may be bound to a tether (as it is added in high concentration). Once a molecule is pulled/diffuses to the surface, it may transiently dissociate a tether and may anneal to the free complementary end of the molecule being sequenced through a pore or to other molecules to form concatemers near the surface of the analysis membrane.

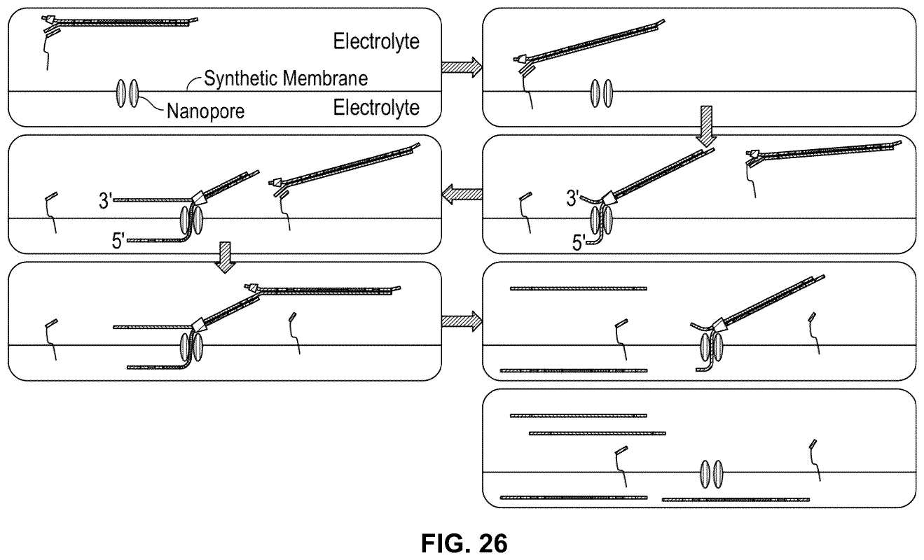

FIG. 26 presents a drawing illustrating an example of a technique for processing a sample with surface-localized annealing. In comparison to true ‘covalent’ concatemers generated through ligation, the analysis techniques may have faster diffusion of molecules to the surface, may not be subject to ligation inefficiencies and may not be susceptible to helicase activity exhaustion. Note that ligation inefficiency in ‘true concatemer’ may lead to sequencing yield loss as the ‘nick’ in the concatemer may sever engagement of the helicase and the pore.

In some embodiments, there may be binding sequences for both the tether oligonucleotide and the adapter on the LP adapter (which may be 3′ ligated). Enabling both to be bound simultaneously may further increase sequencing yield. This may allow the (non-covalent) concatemers to bind multiple tether oligonucleotides, which may improve diffusion to the surface and sequencing yield. Note that the analysis-membrane sequences may need a longer 3′ Y-adapter to allow this operation to be performed.

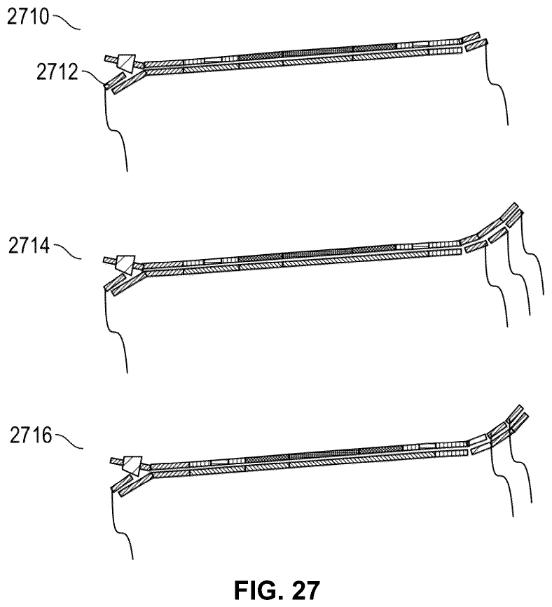

FIG. 27 presents a drawing illustrating an example of a technique for processing a sample with surface-localized annealing. These embodiments may increase tether oligonucleotide annealing sites per library molecule. For example, 3′ added sequence 2712 in the first ligation may match the analysis membrane adapter 3′ end (2710), such that the double-strand DNA library will have a tether oligonucleotide binding site at both ends. Note that more than one copy (2714) of a sequence may be added to enable more than two tethers to be bound per library molecule. An orthogonal 3′ sequence and a splint oligonucleotide may also be used to increase the number of tethers per library without having a long 3′ overhang in the initial LP adapter ligation (2716). This approach may eliminate inter and intra-molecular library annealing. However, this approach may only increase the number of tether bindings. There may not be annealing of library molecules at the pore (to decrease pore dwell time).

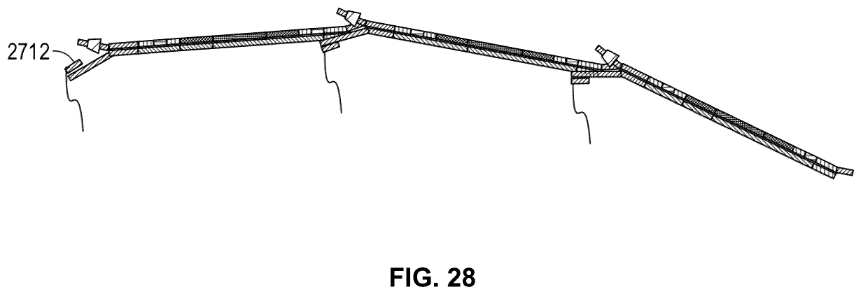

FIG. 28 presents a drawing illustrating an example of a technique for processing a sample with surface-localized annealing. This approach may increase the number of tether oligonucleotides bound to intermolecular annealed libraries. Notably, 3′ added sequence 2712 in the first ligation may have a region matching analysis membrane adapter 3′ end capable of binding to tether oligonucleotide, and a region that can anneal to an exposed portion of the analysis membrane adapter (which may also be 3′ end). Consequently, the double-strand DNA library may have a tether oligonucleotide binding site at both ends. This approach may increase the number of tether oligonucleotides bound in intramolecular annealed libraries, thereby allowing both tether oligonucleotide and another library molecule to be bound in solution or at the pore surface to increase sequencing yield (e.g., by decreasing the dwell time). However, the analysis membrane adapter may need to have an exposed 3′ end that is long enough to be used for both tether and concatemer binding.

In summary, the enrichment and analysis techniques may enable accurate analysis of samples (such as DNA) using an analysis membrane, such as a nanopore.

In the preceding embodiments of the analysis techniques, there may be additional or fewer operations. Furthermore, the order of the operations may be changed, and/or two or more operations may be combined into a single operation.

### Sample

In the present disclosure, a sample can be any biological sample isolated from a subject. Samples can include body tissues, whole blood, platelets, serum, plasma, stool, red blood cells, white blood cells or leucocytes, endothelial cells, tissue biopsies (e.g., biopsies from known or suspected solid tumors), cerebrospinal fluid, synovial fluid, lymphatic fluid, ascites fluid, interstitial or extracellular fluid (e.g., fluid from intercellular spaces), gingival fluid, crevicular fluid, bone marrow, pleural effusions, cerebrospinal fluid, saliva, mucous, sputum, semen, sweat, and/or urine. Samples are preferably body fluids, particularly blood and fractions thereof, and urine. Such samples include nucleic acids shed from tumors. The nucleic acids can include DNA and RNA and can be in double and single-stranded forms. A sample can be in the form originally isolated from a subject or can have been subjected to further processing to remove or add components, such as cells, enrich for one component relative to another, or convert one form of nucleic acid to another, such as RNA to DNA or single-stranded nucleic acids to double-stranded. Thus, e.g., a body fluid for analysis is plasma or serum containing cell-free nucleic acids, e.g., cell-free DNA. In some embodiments, the analysis techniques include obtaining the sample from a subject. Essentially any sample type is optionally utilized. In certain embodiments, e.g., the sample is tissue, blood, plasma, serum, sputum, urine, semen, vaginal fluid, feces, synovial fluid, spinal fluid, saliva, and/or the like. Typically, the subject is a mammalian subject (e.g., a human subject). In some embodiments, the sample is blood. In some embodiments, the sample is plasma. In some embodiments, the sample is serum.

In some embodiments, the sample volume of body fluid taken from a subject depends on the desired read depth for sequenced regions. Exemplary volumes are about 0.4-40 ml, about 5-20 ml, about 10-20 ml. For example, the volume can be about 0.5 ml, about 1 ml, about 5 ml, about 10 ml, about 20 ml, about 30 ml, about 40 ml, or more milliliters. A volume of sampled plasma is typically between about 5 ml to about 20 ml.

The sample can include various amounts of nucleic acid. Typically, the amount of nucleic acid in a given sample is equates with multiple genome equivalents. For example, a sample of about 30 ng DNA can contain about 10,000 (10⁴) haploid human genome equivalents and, in the case of cfDNA, about 200 billion (2×10¹¹) individual polynucleotide molecules. Similarly, a sample of about 100 ng of DNA can contain about 30,000 haploid human genome equivalents and, in the case of cfDNA, about 600 billion individual molecules.

In some embodiments, a sample includes nucleic acids from different sources, e.g., from cells and from cell-free sources (e.g., blood samples, etc.). Typically, a sample includes nucleic acids carrying mutations. For example, a sample optionally includes DNA carrying germline mutations and/or somatic mutations. Alternatively or additionally, a sample includes DNA carrying cancer-associated mutations (e.g., cancer-associated somatic mutations). In some embodiments, the sample includes cell-free DNA (i.e., cfDNA sample). In some embodiments, the cfDNA sample includes circulating tumor nucleic acids.

Exemplary amounts of cell-free nucleic acids in a sample before amplification typically range from about 1 femtogram (fg) to about 1 microgram (μg), e.g., about 1 picogram (pg) to about 200 nanogram (ng), about 1 ng to about 100 ng, or about 10 ng to about 1000 ng. In some embodiments, a sample includes up to about 600 ng, up to about 500 ng, up to about 400 ng, up to about 300 ng, up to about 200 ng, up to about 100 ng, up to about 50 ng, or up to about 20 ng of cell-free nucleic acid molecules. Optionally, the amount is at least about 1 fg, at least about 10 fg, at least about 100 fg, at least about 1 pg, at least about 10 pg, at least about 100 pg, at least about 1 ng, at least about 10 ng, at least about 100 ng, at least about 150 ng, or at least about 200 ng of cell-free nucleic acid molecules. In certain embodiments, the amount is up to about 1 fg, about 10 fg, about 100 fg, about 1 pg, about 10 pg, about 100 pg, about 1 ng, about 10 ng, about 100 ng, about 150 ng, or about 200 ng of cell-free nucleic acid molecules. In some embodiments, the analysis techniques include obtaining between about 1 fg to about 200 ng cell-free nucleic acid molecules from samples. In certain embodiments, the analysis techniques include obtaining between about 5 ng to about 30 ng of cell-free nucleic acid molecules from samples. In certain embodiments, the analysis techniques include obtaining between about 5 ng to about 100 ng of cell-free nucleic acid molecules from samples. In certain embodiments, the analysis techniques include obtaining between about 5 ng to about 150 ng of cell-free nucleic acid molecules from samples. In certain embodiments, the analysis techniques include obtaining between about 5 ng to about 200 ng of cell-free nucleic acid molecules from samples. In some embodiments, the amount is up to about 100 ng of cell-free nucleic acid molecules from samples. In some embodiments, the amount is up to about 150 ng of cell-free nucleic acid molecules from samples. In some embodiments, the amount is up to about 200 ng of cell-free nucleic acid molecules from samples. In some embodiments, the amount is up to about 250 ng of cell-free nucleic acid molecules from samples. In some embodiments, the amount is up to about 300 ng of cell-free nucleic acid molecules from samples. In some embodiments, the analysis techniques include obtaining between about 1 fg to about 200 ng cell-free nucleic acid molecules from samples.

Cell-free nucleic acids typically have a size distribution of between about 100 nucleotides in length and about 500 nucleotides in length, with molecules of about 110 nucleotides in length to about 230 nucleotides in length representing about 90% of molecules in the sample, with a mode of about 168 nucleotides in length and a second minor peak in a range between about 240 to about 440 nucleotides in length. In certain embodiments, cell-free nucleic acids are from about 160 to about 180 nucleotides in length, or from about 320 to about 360 nucleotides in length, or from about 440 to about 480 nucleotides in length.

In some embodiments, cell-free nucleic acids are isolated from bodily fluids through a partitioning operation in which cell-free nucleic acids, as found in solution, are separated from intact cells and other non-soluble components of the bodily fluid. In some of these embodiments, partitioning includes analysis techniques such as centrifugation or filtration. Alternatively, cells in bodily fluids are lysed, and cell-free and cellular nucleic acids processed together. Generally, after addition of buffers and wash operations, cell-free nucleic acids are precipitated with, e.g., an alcohol. In certain embodiments, additional clean-up operations are used, such as silica-based columns to remove contaminants or salts. Non-specific bulk carrier nucleic acids, e.g., are optionally added throughout the reaction to optimize certain aspects of the exemplary procedure, such as yield. After such processing, samples typically include various forms of nucleic acids including double-stranded DNA, single-stranded DNA and/or single-stranded RNA. Optionally, single stranded DNA and/or single stranded RNA are converted to double stranded forms so that they are included in subsequent processing and analysis operations.

In some embodiments, the nucleic acid molecules of the sample are subjected to a conversion process to detect methylation status of a nucleobase (e.g., 5-methylcytosine or 5-hydroxymethylcytosine). Such conversion methods may include, but are not limited to bisulfite conversion, oxidative bisulfite conversion, Tet-assisted bisulfite conversion, APOBEC-couple epigenetic conversion, enzymatic methyl-seq (EM-seq), single-enzyme 5-methylcytosine sequencing (SEM-seq), and Tet-assisted pyridine borane sequencing (TAPS). In some embodiments, the conversion procedure converts modified nucleosides, such as in DM-seq (as described in WO2023/288222A1).

In some embodiments, methods herein comprise contacting DNA with a methylation-sensitive nuclease, thereby degrading DNA comprising unmethylated sequences or sequences having low levels of methylation. In some such embodiments, the methylation-sensitive nuclease is a methylation-sensitive restriction enzyme (MSRE), thereby degrading DNA comprising an unmethylated recognition site of the MSRE. Methylation-sensitive nucleases can thus be used in methods herein comprising one or more steps that deplete unmodified or unmethylated sequences, such as those that are prevalent in cfDNA from a subject.

In some embodiments, methods herein comprise contacting DNA with a methylation-dependent nuclease, thereby degrading DNA comprising methylated sequences or sequences having high levels of methylation. In some such embodiments, the methylation-dependent nuclease is a methylation-dependent restriction enzyme (MDRE), thereby degrading DNA comprising a methylated recognition site of the MDRE. Methylation-dependent nucleases can thus be used in methods herein comprising one or more steps that deplete modified or methylated sequences, such as those that are prevalent in cfDNA from a subject.

### Nucleic Acid Tags

In some embodiments, the nucleic acid molecules (from the sample of polynucleotides) may be tagged with sample indexes and/or molecular barcodes (referred to generally as ‘tags’). Tags may be incorporated into or otherwise joined to adapters by chemical synthesis, ligation (e.g., blunt-end ligation or sticky-end ligation), or overlap extension PCR, among other methods. Such adapters may be ultimately joined to the target nucleic acid molecule. In other embodiments, one or more rounds of amplification cycles (e.g., PCR amplification) are generally applied to introduce sample indexes to a nucleic acid molecule using conventional nucleic acid amplification methods. The amplifications may be conducted in one or more reaction mixtures (e.g., a plurality of microwells in an array). Molecular barcodes and/or sample indexes may be introduced simultaneously, or in any sequential order. In some embodiments, molecular barcodes and/or sample indexes are introduced prior to and/or after sequence capturing operations are performed. In some embodiments, only the molecular barcodes are introduced prior to probe capturing and the sample indexes are introduced after sequence capturing operations are performed. In some embodiments, both the molecular barcodes and the sample indexes are introduced prior to performing probe-based capturing operations. In some embodiments, the sample indexes are introduced after sequence capturing operations are performed. In some embodiments, molecular barcodes are incorporated to the nucleic acid molecules (e.g., cfDNA molecules) in a sample through adapters via ligation (e.g., blunt-end ligation or sticky-end ligation). In some embodiments, sample indexes are incorporated to the nucleic acid molecules (e.g., cfDNA molecules) in a sample through overlap extension PCR. Typically, sequence capturing protocols involve introducing a single-stranded nucleic acid molecule complementary to a targeted nucleic acid sequence, e.g., a coding sequence of a genomic region and mutation of such region is associated with a cancer type.

In some embodiments, the tags may be located at one end or at both ends of the sample nucleic acid molecule. In some embodiments, tags are predetermined or random or semi-random sequence oligonucleotides. In some embodiments, the tags may be less than about 500, 200, 100, 50, 20, 10, 9, 8, 7, 6, 5, 4, 3, 2, or 1 nucleotides in length. The tags may be linked to sample nucleic acids randomly or non-randomly.

In some embodiments, each sample is uniquely tagged with a sample index or a combination of sample indexes. In some embodiments, each nucleic acid molecule of a sample or sub-sample is uniquely tagged with a molecular barcode or a combination of molecular barcodes. In other embodiments, a plurality of molecular barcodes may be used such that molecular barcodes are not necessarily unique to one another in the plurality (e.g., non-unique molecular barcodes). In these embodiments, molecular barcodes are generally attached (e.g., by ligation) to individual molecules such that the combination of the molecular barcode and the sequence it may be attached to creates a unique sequence that may be individually tracked. Detection of non-uniquely tagged molecular barcodes in combination with endogenous sequence information (e.g., the beginning (start) and/or end (stop) portions corresponding to the sequence of the original nucleic acid molecule in the sample, sub-sequences of sequence reads at one or both ends, length of sequence reads, and/or length of the original nucleic acid molecule in the sample) typically allows for the assignment of a unique identity to a particular molecule. The length, or number of base pairs, of an individual sequence read are also optionally used to assign a unique identity to a given molecule. As described herein, fragments from a single strand of nucleic acid having been assigned a unique identity, may thereby permit subsequent identification of fragments from the parent strand, and/or a complementary strand.

In some embodiments, molecular barcodes are introduced at an expected ratio of a set of identifiers (e.g., a combination of unique or non-unique molecular barcodes) to molecules in a sample. One example format uses from about 2 to about 1,000,000 different molecular barcodes, or from about 5 to about 150 different molecular barcodes, or from about 20 to about 50 different molecular barcodes, ligated to both ends of a target molecule. Alternatively, from about 25 to about 1,000,000 different molecular barcodes may be used. For example, 20-50×20-50 molecular barcodes can be used. In some embodiments, 20-50 different molecular barcodes can be used. In some embodiments, 5-100 different molecular barcodes can be used. In some embodiments, 5-150 molecular barcodes can be used. In some embodiments, 5-200 different molecular barcodes can be used. Such numbers of identifiers are typically sufficient for different molecules having the same start and stop points to have a high probability (e.g., at least 94%, 99.5%, 99.99%, or 99.999%) of receiving different combinations of identifiers. In some embodiments, about 80%, about 90%, about 95%, or about 99% of molecules have the same combinations of molecular barcodes.

In some embodiments, the assignment of unique or non-unique molecular barcodes in reactions is performed using methods and systems described in, e.g., U.S. Patent Application Nos. 20010053519, 20030152490, and 20110160078, and U.S. Pat. Nos. 6,582,908, 7,537,898, 9,598,731, and 9,902,992, each of which is hereby incorporated by reference in its entirety. Alternatively, in some embodiments, different nucleic acid molecules of a sample may be identified using only endogenous sequence information (e.g., start and/or stop positions, sub-sequences of one or both ends of a sequence, and/or lengths).

### Amplification

Sample nucleic acids flanked by adapters are typically amplified by PCR and other amplification methods using nucleic acid primers binding to primer binding sites in adapters flanking a molecule of DNA to be amplified. In some embodiments, amplification methods involve cycles of extension, denaturation and annealing resulting from thermocycling, or can be isothermal as, e.g., in transcription mediated amplification. Other amplification exemplary methods that are optionally utilized, include the ligase chain reaction, strand displacement amplification, nucleic acid sequence-based amplification, and self-sustained sequence-based replication, among other approaches.

One or more rounds of amplification cycles are generally applied to introduce molecular barcodes and/or sample indexes to a nucleic acid molecule using conventional nucleic acid amplification methods. The amplifications are typically conducted in one or more reaction mixtures. Molecular barcodes and sample indexes are optionally introduced simultaneously, or in any sequential order. In some embodiments, molecular barcodes and sample indexes are introduced prior to and/or after sequence capturing operations are performed. In some embodiments, only the molecular barcodes are introduced prior to probe capturing and the sample indexes are introduced after sequence capturing operations are performed. In certain embodiments, both the molecular barcodes and the sample indexes are introduced prior to performing probe-based capturing operations. In some embodiments, the sample indexes are introduced after sequence capturing operations are performed. Typically, sequence capturing protocols involve introducing a single-stranded nucleic acid molecule complementary to a targeted nucleic acid sequence, e.g., a coding sequence of a genomic region and mutation of such region is associated with a cancer type. Alternatively or additionally, typically the amplification reactions generate a plurality of non-uniquely or uniquely tagged nucleic acid amplicons with molecular barcodes and sample indexes at size ranging from about 200 nucleotides (nt) to about 700 nt, from 250 nt to about 350 nt, or from about 320 nt to about 550 nt. In some embodiments, the amplicons have a size of about 300 nt. In some embodiments, the amplicons have a size of about 500 nt.

### Enrichment

Sequences can be enriched prior to sequencing. Enrichment can be performed for specific target regions or nonspecifically (‘target sequences’). In some embodiments, target regions of interest may be enriched with capture probes (‘baits’) selected for one or more bait set panels using a differential tiling and capture technique. A differential tiling and capture scheme uses bait sets of different relative concentrations to differentially tile (e.g., at different ‘resolutions’) across genomic regions associated with baits, subject to a set of constraints (e.g., sequencer constraints such as sequencing load, utility of each bait, etc.), and capture them at a desired level for downstream sequencing. These targeted genomic regions of interest may include natural or synthetic nucleotide sequences of the nucleic acid construct. In some embodiments, biotin-labeled beads with probes to one or more regions of interest can be used to capture target sequences, optionally followed by amplification of those regions, to enrich for the regions of interest.

Sequence capture may include the use of oligonucleotide probes that hybridize to the target sequence. A probe set strategy can involve tiling the probes across a region of interest. Such probes can be, e.g., about 60 to 120 bases long. The set can have a depth of about 2×, 3×, 4×, 5×, 6×, 8×, 9×, 10×, 15×, 20×, 50×, or more than 50×. The effectiveness of sequence capture depends, in part, on the length of the sequence in the target molecule that is complementary (or nearly complementary) to the sequence of the probe.

In some embodiments, the plurality of genomic regions includes genetic variants found in the Catalogue of Somatic Mutations in Cancer (COSMIC), The Cancer Genome Atlas (TCGA), or the Exome Aggregation Consortium (ExAC). In some cases, genetic variants may belong to a pre-defined set of clinically actionable variants. For example, such variants may be found in various databases of variants whose presence in a sample of a subject have been shown to correlate with or be indicative of a disease or disorder (e.g., cancer) in the subject. Such databases of variants may include, e.g., COSMIC, TCGA, and the ExAC. A pre-defined set of such catalogued variants may be designated for further bioinformatics analysis due to their relevance to clinical decision-making (e.g., diagnosis, prognosis, treatment selection, targeted treatment, treatment monitoring, monitoring for recurrence, etc.). Such a pre-defined set may be determined based on, e.g., analysis of clinical samples (e.g., of patient cohorts with known presence or absence of a disease or disorder) as well as annotation information from public databases and clinical literature.

### Sequencing

Sample nucleic acids flanked by adapters with or without prior amplification can be subject to sequencing. In some embodiments, the sequencing methods include, e.g., Sanger sequencing, high-throughput sequencing, pyrosequencing, sequencing-by-synthesis, single-molecule sequencing, nanopore sequencing, semiconductor sequencing, sequencing-by-ligation, sequencing-by-hybridization, RNA-Seq (from Illumina), Digital Gene Expression (from Helicos BioSciences of Cambridge, Massachusetts), Next generation sequencing, Single Molecule Sequencing by Synthesis or SMSS (from Helicos), massively-parallel sequencing, Clonal Single Molecule Array (from Solexa, a division of Illumina, Inc. of San Diego, California), shotgun sequencing, Ion Torrent, Oxford Nanopore, Roche Genia, Maxim-Gilbert sequencing, primer walking, sequencing using PacBio, SOLID, Ion Torrent, or Nanopore platforms. Sequencing reactions can be performed in a variety of sample processing units, which may multiple lanes, multiple channels, multiple wells, or other mean of processing multiple sample sets substantially simultaneously. Sample processing unit can also include multiple sample chambers to enable processing of multiple runs simultaneously.

In some embodiments, the sequencing comprises the use of a membrane, such as a nanopore. One of the advantages of nanopore sequencing is the ability to detect methylation directly from the nucleic acid molecules in a sample without the need to expose the nucleic acids to chemical or enzymatic techniques, such as bisulfite conversion or EM-Seq.

The sequencing reactions can be performed on one or more nucleic acid fragment types or regions known to contain markers of cancer or other diseases. The sequencing reactions can also be performed on any nucleic acid fragment present in the sample. The sequence reactions may be performed on at least about 5%, 10%, 15%, 20%, 25%, 30%, 40%, 50%, 60%, 70%, 80%, 90%, 95%, 99%, 99.9% or 100% of the genome. In other cases, sequence reactions may be performed on less than about 5%, 10%, 15%, 20%, 25%, 30%, 40%, 50%, 60%, 70%, 80%, 90%, 95%, 99%, 99.9% or 100% of the genome.

Simultaneous sequencing reactions may be performed using multiplex sequencing techniques. In some cases, cell free polynucleotides may be sequenced with at least 1000, 2000, 3000, 4000, 5000, 6000, 7000, 8000, 9000, 10000, 50000, or 100,000 sequencing reactions. In other cases, cell free polynucleotides may be sequenced with less than 1000, 2000, 3000, 4000, 5000, 6000, 7000, 8000, 9000, 10000, 50000, or 100,000 sequencing reactions. Sequencing reactions may be performed sequentially or simultaneously. Subsequent data analysis may be performed on all or part of the sequencing reactions. In some cases, data analysis may be performed on at least 1000, 2000, 3000, 4000, 5000, 6000, 7000, 8000, 9000, 10000, 50000, or 100,000 sequencing reactions. In other cases, data analysis may be performed on less than 1000, 2000, 3000, 4000, 5000, 6000, 7000, 8000, 9000, 10000, 50000, or 100,000 sequencing reactions. An exemplary read depth is 1000-50000 reads per locus (base). In some embodiments, read depth can be greater than 50000 reads per locus (base).

### Analysis

Sequencing according to embodiments of the disclosed analysis techniques generates a plurality of sequencing reads or reads. Sequencing reads or reads according to the disclosed analysis techniques generally include sequences of nucleotide data less than about 150 bases in length, or less than about 90 bases in length. In certain embodiments, reads are between about 80 and about 90 bases, e.g., about 85 bases in length. In some embodiments, methods of the disclosed analysis techniques are applied to very short reads, i.e., less than about 50 or about 30 bases in length. Sequencing read data can include the sequence data as well as meta information. Sequence read data can be stored in any suitable file format including, e.g., VCF files, FASTA files or FASTQ files.

FASTA is originally a computer program for searching sequence databases and the name FASTA has come to also refer to a standard file format. See Pearson & Lipman, 1988, “Improved tools for biological sequence comparison,” PNAS 85:2444-2448. A sequence in FASTA format begins with a single-line description, followed by lines of sequence data. The description line is distinguished from the sequence data by a greater-than (‘>’) symbol in the first column. The word following the ‘>’ symbol is the identifier of the sequence, and the rest of the line is the description (both are optional). There should be no space between the ‘>’ and the first letter of the identifier. It is recommended that all lines of text be shorter than 80 characters. The sequence ends if another line starting with a ‘>’ appears; this indicates the start of another sequence.

The FASTQ format is a text-based format for storing both a biological sequence (usually nucleotide sequence) and its corresponding quality scores. It is similar to the FASTA format but with quality scores following the sequence data. Both the sequence letter and quality score are encoded with a single ASCII character for brevity. The FASTQ format is a de facto standard for storing the output of high throughput sequencing instruments such as the Illumina Genome Analyzer, as described by, e.g., Cock et al. (“The Sanger FASTQ file format for sequences with quality scores, and the Solexa/Illumina FASTQ variants,” Nucleic Acids Res 38 (6): 1767-1771, 2009), which is hereby incorporated by reference in its entirety.

For FASTA and FASTQ files, meta information includes the description line and not the lines of sequence data. In some embodiments, for FASTQ files, the meta information includes the quality scores. For FASTA and FASTQ files, the sequence data begins after the description line and is present typically using some subset of IUPAC ambiguity codes optionally with ‘-’. In a preferred embodiment, the sequence data will use the A, T, C, G, and N characters, optionally including ‘-’ or U as-needed (e.g., to represent gaps or uracil).

In some embodiments, the at least one master sequence read file and the output file are stored as plain text files (e.g., using encoding such as ASCII; ISO/IEC 646; EBCDIC; UTF-8; or UTF-16). A computer system provided by the disclosed analysis techniques may include a text editor program capable of opening the plain text files. A text editor program may refer to a computer program capable of presenting contents of a text file (such as a plain text file) on a computer screen, allowing a human to edit the text (e.g., using a monitor, keyboard, and mouse). Exemplary text editors include, without limit, Microsoft Word, emacs, pico, vi, BBEdit, and TextWrangler. Preferably, the text editor program is capable of displaying the plain text files on a computer screen, showing the meta information and the sequence reads in a human-readable format (e.g., not binary encoded but instead using alphanumeric characters as they may be used in print human writing).

While methods have been discussed with reference to FASTA or FASTQ files, methods and systems of the disclosed analysis techniques may be used to compress any suitable sequence file format including, e.g., files in the Variant Call Format (VCF) format. A typical VCF file will include a header section and a data section. The header contains an arbitrary number of meta-information lines, each starting with characters ‘##’, and a TAB delimited field definition line starting with a single ‘#’ character. The field definition line names eight mandatory columns and the body section contains lines of data populating the columns defined by the field definition line. The VCF format is described by Danecek et al. (“The variant call format and VCFtools,” Bioinformatics 27 (15): 2156-2158, 2011), which is hereby incorporated by reference in its entirety. The header section may be treated as the meta information to write to the compressed files and the data section may be treated as the lines, each of which will be stored in a master file only if unique.

Certain embodiments of the disclosed analysis techniques provide for the assembly of sequencing reads. In assembly by alignment, e.g., the sequencing reads are aligned to each other or aligned to a reference sequence. By aligning each read, in turn to a reference genome, all of the reads are positioned in relationship to each other to create the assembly. In addition, aligning or mapping the sequencing read to a reference sequence can also be used to identify variant sequences within the sequencing read. Identifying variant sequences can be used in combination with the methods and systems described herein to further aid in the diagnosis or prognosis of a disease or condition, or for guiding treatment decisions.

In some embodiments, any or all of the operations are automated. Alternatively, methods of the disclosed analysis techniques may be embodied wholly or partially in one or more dedicated programs, e.g., each optionally written in a compiled language such as C++ then compiled and distributed as a binary. Methods of the disclosed analysis techniques may be implemented wholly or in part as modules within, or by invoking functionality within, existing sequence analysis platforms. In certain embodiments, methods of the disclosed analysis techniques include a number of operations that are all invoked automatically responsive to a single starting cue (e.g., one or a combination of triggering events sourced from human activity, another computer program, or a machine). Thus, the disclosed analysis techniques provide methods in which any or the operations or any combination of the operations can occur automatically responsive to a cue. Automatically generally means without intervening human input, influence, or interaction (i.e., responsive only to original or pre-cue human activity).

The system also encompasses various forms of output, which includes an accurate and sensitive interpretation of the subject nucleic acid. The output of retrieval can be provided in the format of a computer file. In certain embodiments, the output is a FASTA file, FASTQ file, or VCF file. Output may be processed to produce a text file, or an XML file containing sequence data such as a sequence of the nucleic acid aligned to a sequence of the reference genome. In other embodiments, processing yields output containing coordinates or a string describing one or more mutations in the subject nucleic acid relative to the reference genome. Alignment strings may include Simple UnGapped Alignment Report (SUGAR), Verbose Useful Labeled Gapped Alignment Report (VULGAR), and Compact Idiosyncratic Gapped Alignment Report (CIGAR) (Ning et al., Genome Research 11 (10): 1725-9, 2001, which is hereby incorporated by reference in its entirety). These strings are implemented, e.g., in the Exonerate sequence alignment software from the European Bioinformatics Institute (Hinxton, United Kingdom).

In some embodiments, a sequence alignment is produced (such as, e.g., a sequence alignment map or SAM, or binary alignment map or BAM file) including a CIGAR string (the SAM format is described, e.g., by Li et al., “The Sequence Alignment/Map format and SAMtools,” Bioinformatics, 25 (16): 2078-9, 2009, which is hereby incorporated by reference in its entirety). In some embodiments, CIGAR displays or includes gapped alignments one-per-line. CIGAR is a compressed pairwise alignment format reported as a CIGAR string. A CIGAR string is useful for representing long (e.g., genomic) pairwise alignments. A CIGAR string is used in SAM format to represent alignments of reads to a reference genome sequence.

A CIGAR string follows an established motif. Each character is preceded by a number, giving the base counts of the event. Characters used can include M, I, D, N, and S (M=match; I=insertion; D=deletion; N=gap; S=substitution). The CIGAR string defines the sequence of matches/mismatches and deletions (or gaps). For example, the CIGAR string 2MD3M2D2M will mean that the alignment contains 2 matches, 1 deletion (number 1 is omitted in order to save some space), 3 matches, 2 deletions and 2 matches.

In some embodiments, a nucleic acid population is prepared for sequencing by enzymatically forming blunt-ends on double-stranded nucleic acids with single-stranded overhangs at one or both ends. In these embodiments, the population is typically treated with an enzyme having a 5′-3′ DNA polymerase activity and a 3′-5′ exonuclease activity in the presence of the nucleotides (e.g., A, C, G and T or U) in the form of dNTPs. Exemplary enzymes or catalytic fragments thereof that are optionally used include Klenow large fragment and T4 polymerase. At 5′ overhangs, the enzyme typically extends the recessed 3′ end on the opposing strand until it is flush with 5′ end to produce a blunt end. At 3′ overhangs, the enzyme generally digests from 3′ end up to and sometimes beyond 5′ end of the opposing strand. If this digestion proceeds beyond 5′ end of the opposing strand, the gap can be filled in by an enzyme having the same polymerase activity that is used for 5′ overhangs. The formation of blunt-ends on double-stranded nucleic acids facilitates, e.g., the attachment of adapters and subsequent amplification.

In some embodiments, nucleic acid populations are subject to additional processing, such as the conversion of single-stranded nucleic acids to double-stranded and/or conversion of RNA to DNA. These forms of nucleic acid are also optionally linked to adapters and amplified.

With or without prior amplification, nucleic acids subject to the process of forming blunt-ends described above, and optionally other nucleic acids in a sample, can be sequenced to produce sequenced nucleic acids. A sequenced nucleic acid can refer either to the sequence of a nucleic acid (i.e., sequence information) or a nucleic acid whose sequence has been determined. Sequencing can be performed so as to provide sequence data of individual nucleic acid molecules in a sample either directly or indirectly from a consensus sequence of amplification products of an individual nucleic acid molecule in the sample.

In some embodiments, double-stranded nucleic acids with single-stranded overhangs in a sample after blunt-end formation are linked at both ends to adapters including molecular barcodes, and the sequencing determines nucleic acid sequences as well as molecular barcodes introduced by the adapters. The blunt-end molecules of DNA are optionally ligated to a blunt end of an at least partially double-stranded adapter (e.g., a Y shaped or bell-shaped adapter). Alternatively, blunt ends of sample nucleic acids and adapters can be tailed with complementary nucleotides to facilitate ligation (for e.g., sticky end ligation).

The nucleic acid sample is typically contacted with a sufficient number of adapters that there is a low probability (e.g., <1 or <0.1%) that any two copies of the same nucleic acid receive the same combination of adapter barcodes (i.e., molecular barcodes) from the adapters linked at both ends. The use of adapters in this manner permits identification of families of nucleic acid sequences with the same start and stop points on a reference nucleic acid and linked to the same combination of molecular barcodes. Such a family represents sequences of amplification products of a nucleic acid in the sample before amplification. The sequences of family members can be compiled to derive consensus nucleotide(s) or a complete consensus sequence for a nucleic acid molecule in the original sample, as modified by blunt end formation and adapter attachment. In other words, the nucleotide occupying a specified position of a nucleic acid in the sample is determined to be the consensus of nucleotides occupying that corresponding position in family member sequences. Families can include sequences of one or both strands of a double-stranded nucleic acid. If members of a family include sequences of both strands from a double-stranded nucleic acid, sequences of one strand are converted to their complement for purposes of compiling all sequences to derive consensus nucleotide(s) or sequences. Some families include only a single member sequence. In this case, this sequence can be taken as the sequence of a nucleic acid in the sample before amplification. Alternatively, families with only a single member sequence can be eliminated from subsequent analysis.

Nucleotide variations in sequenced nucleic acids can be determined by comparing sequenced nucleic acids with a reference sequence. The reference sequence is often a known sequence, e.g., a known whole or partial genome sequence from a subject (e.g., a whole genome sequence of a human subject). The reference sequence can be, e.g., hG19 or hG38. The sequenced nucleic acids can represent sequences determined directly for a nucleic acid in a sample, or a consensus of sequences of amplification products of such a nucleic acid, as described above. A comparison can be performed at one or more designated positions on a reference sequence. A subset of sequenced nucleic acids can be identified including a position corresponding with a designated position of the reference sequence when the respective sequences are maximally aligned. Within such a subset it can be determined which, if any, sequenced nucleic acids include a nucleotide variation at the designated position, and optionally which if any, include a reference nucleotide (i.e., same as in the reference sequence). If the number of sequenced nucleic acids in the subset including a nucleotide variant exceeding a selected threshold, then a variant nucleotide can be called at the designated position. The threshold can be a simple number, such as at least 1, 2, 3, 4, 5, 6, 7, 9, or 10 sequenced nucleic acids within the subset including the nucleotide variant or it can be a ratio, such as a least 0.5, 1, 2, 3, 4, 5, 10, 15, or 20 of sequenced nucleic acids within the subset that include the nucleotide variant, among other possibilities. The comparison can be repeated for any designated position of interest in the reference sequence. Sometimes a comparison can be performed for designated positions occupying at least about 20, 100, 200, or 300 contiguous positions on a reference sequence, e.g., about 20-500, or about 50-300 contiguous positions.

Additional details regarding nucleic acid sequencing, including the formats and applications described herein are also provided in, e.g., Levy et al., Annual Review of Genomics and Human Genetics, 17:95-115 (2016), Liu et al., J. of Biomedicine and Biotechnology, Volume 2012, Article ID 251364:1-11 (2012), Voelkerding et al., Clinical Chem., 55:641-658 (2009), MacLean et al., Nature Rev. Microbiol., 7:287-296 (2009), Astier et al., J Am Chem Soc., 128 (5): 1705-10 (2006), U.S. Pat. Nos. 6,210,891, 6,258,568, 6,833,246, 7,115,400, 6,969,488, 5,912,148, 6,130,073, 7,169,560, 7,282,337, 7,482,120, 7,501,245, 6,818,395, 6,911,345, 7,501,245, 7,329,492, 7,170,050, 7,302,146, 7,313,308, and 7,476,503, which are each incorporated by reference in their entirety.

### Applications

### Cancer and Other Diseases

Typically, the disease under consideration is a type of cancer. Non-limiting examples of such cancers include biliary tract cancer, bladder cancer, transitional cell carcinoma, urothelial carcinoma, brain cancer, gliomas, astrocytomas, breast carcinoma, metaplastic carcinoma, cervical cancer, cervical squamous cell carcinoma, rectal cancer, colorectal carcinoma, colon cancer, hereditary nonpolyposis colorectal cancer, colorectal adenocarcinomas, gastrointestinal stromal tumors (GISTs), endometrial carcinoma, endometrial stromal sarcomas, esophageal cancer, esophageal squamous cell carcinoma, esophageal adenocarcinoma, ocular melanoma, uveal melanoma, gallbladder carcinomas, gallbladder adenocarcinoma, renal cell carcinoma, clear cell renal cell carcinoma, transitional cell carcinoma, urothelial carcinomas, Wilms tumor, leukemia, acute lymphocytic leukemia (ALL), acute myeloid leukemia (AML), chronic lymphocytic leukemia (CLL), chronic myeloid leukemia (CML), chronic myelomonocytic leukemia (CMML), liver cancer, liver carcinoma, hepatoma, hepatocellular carcinoma, cholangiocarcinoma, hepatoblastoma, Lung cancer, non-small cell lung cancer (NSCLC), mesothelioma, B-cell lymphomas, non-Hodgkin lymphoma, diffuse large B-cell lymphoma, Mantle cell lymphoma, T cell lymphomas, non-Hodgkin lymphoma, precursor T-lymphoblastic lymphoma/leukemia, peripheral T cell lymphomas, multiple myeloma, nasopharyngeal carcinoma (NPC), neuroblastoma, oropharyngeal cancer, oral cavity squamous cell carcinomas, osteosarcoma, ovarian carcinoma, pancreatic cancer, pancreatic ductal adenocarcinoma, pseudopapillary neoplasms, acinar cell carcinomas, Prostate cancer, prostate adenocarcinoma, skin cancer, melanoma, malignant melanoma, cutaneous melanoma, small intestine carcinomas, stomach cancer, gastric carcinoma, gastrointestinal stromal tumor (GIST), uterine cancer, or uterine sarcoma.

Non-limiting examples of other genetic-based diseases, disorders, or conditions that are optionally evaluated using the methods and systems disclosed herein include achondroplasia, alpha-1 antitrypsin deficiency, antiphospholipid syndrome, autism, autosomal dominant polycystic kidney disease, Charcot-Marie-Tooth (CMT), cri du chat, Crohn's disease, cystic fibrosis, Dercum disease, down syndrome. Duane syndrome, Duchenne muscular dystrophy, Factor V Leiden thrombophilia, familial hypercholesterolemia, familial mediterranean fever, fragile X syndrome, Gaucher disease, hemochromatosis, hemophilia, holoprosencephaly. Huntington's disease, Klinefelter syndrome, Marfan syndrome, myotonic dystrophy, neurofibromatosis. Noonan syndrome, osteogenesis imperfecta. Parkinson's disease, phenylketonuria, Poland anomaly, porphyria, progeria, retinitis pigmentosa, severe combined immunodeficiency (scid), sickle cell disease, spinal muscular atrophy, Tay-Sachs, thalassemia, trimethylaminuria, Turner syndrome, velocardiofacial syndrome, WAGR syndrome. Wilson disease, or the like.

In certain embodiments, the methods disclosed herein relate to identifying and administering therapies, such as customized therapies, to patients or subjects. In some embodiments, determination of the presence or absence or levels of polypeptides and/or genetic or epigenomic variations, facilitates selection of appropriate treatment. In some embodiments, the patient or subject has a given disease, disorder or condition, e.g., any of the cancers or other conditions described elsewhere herein. Essentially any cancer therapy (e.g., surgical therapy, radiation therapy, chemotherapy, immunotherapy, and/or the like) may be included as part of these methods.

In certain embodiments, the therapies can include one or more of treatments for target therapies, including abemaciclib (Verzenio), abiraterone acetate (Zytiga), acalabrutinib (Calquence), adagrasib (Krazati), ado-trastuzumab emtansine (Kadcyla), afatinib dimaleate (Gilotrif), alectinib (Alecensa), alemtuzumab (Campath), alitretinoin (Panretin), alpelisib (Piqray), amivantamab-vmjw (Rybrevant), anastrozole (Arimidex), apalutamide (Erleada), asciminib hydrochloride (Scemblix), atezolizumab (Tecentriq), atezolizumab (Tecentriq), avapritinib (Ayvakit), avelumab (Bavencio), axicabtagene ciloleucel (Yescarta), axitinib (Inlyta), belinostat (Beleodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib-s-malate (Cabometyx), cabozantinib-s-malate (Cometriq), capmatinib hydrochloride (Tabrecta), carfilzomib (Kyprolis), cemiplimab-rwlc (Libtayo), ceritinib (Zykadia), cetuximab (Erbitux), ciltacabtagene autoleucel (Carvykti), cobimetinib fumarate (Cotellic), copanlisib hydrochloride (Aliqopa), crizotinib (Xalkori), dabrafenib (Tafmlar), dabrafenib mesylate (Tafmlar), dacomitinib (Vizimpro), daratumumab (Darzalex), daratumumab and hyaluronidase-fihj (Darzalex Faspro), darolutamide (Nubeqa), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), elacestrant dihydrochloride (Orserdu), clotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib hydrochloride (Tarceva), everolimus (Afinitor), exemestane (Aromasin), fam-trastuzumab deruxtecan-nxki (Enhertu), fam-trastuzumab deruxtecan-nxki (Enhertu), fedratinib hydrochloride (Inrebic), fulvestrant (Faslodex), futibatinib (Lytgobi), gefitinib (Iressa), gemtuzumab ozogamicin (Mylotarg), gilteritinib fumarate (Xospata), glasdegib maleate (Daurismo), ibritumomab tiuxctan (Zevalin), ibrutinib (Imbruvica), idecabtagene vicleucel (Abecma), idelalisib (Zydelig), imatinib mesylate (Gleevec), infigratinib phosphate (Truseltiq), inotuzumab ozogamicin (Besponsa), iobenguane 1 131 (Azcdra), ipilimumab (Yervoy), isatuximab-irfc (Sarclisa), ivosidenib (Tibsovo), ixazomib citrate (Ninlaro), lanreotide acetate (SomatulineDepot), lapatinib ditosylate (Tykerb), larotrectinib sulfate (Vitrakvi), lenvatinib mesylate (Lenvima), letrozole (Femara), lisocabtagene maraleucel (Breyanzi), loncastuximab tesirine-lpyl (Zynlonta), lorlatinib (Lorbrena), lutetium Lu 177 vipivotide tetraxetan (Pluvicto), lutetium Lu 177-dotatate (Lutathera), margetuximab-cmkb (Margenza), midostaurin (Rydapt), mirvetuximab soravtansine-gynx (Elahere), mobocertinib succinate (Exkivity), mogamulizumab-kpkc (Poteligco), mosunetuzumab-axgb (Lunsumio), moxetumomab pasudotox-tdfk (Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), nivolumab and relatlimab-rmbw (Opdualag), obinutuzumab (Gazyva), ofatumumab (Arzerra), olaparib (Lynparza), olutasidenib (Rezlidhia), osimertinib mesylate (Tagrisso), pacritinib citrate (Vonjo), palbociclib (Ibrance), panitumumab (Vectibix), pazopanib hydrochloride (Votrient), pembrolizumab (Keytruda), pemigatinib (Pemazyrc), pertuzumab (Perjeta), pertuzumab, trastuzumab, and hyaluronidase-zzxf (Phesgo), pexidartinib hydrochloride (Turalio), pirtobrutinib (Jaypirca), polatuzumab vedotin-piiq (Polivy), ponatinib hydrochloride (Iclusig), pralatrexate (Folotyn), pralsctinib (Gavreto), radium 223 dichloride (Xofigo), ramucirumab (Cyramza), regorafenib (Stivarga), retifanlimab-dlwr (Zynyz), ribociclib (Kisqali), ripretinib (Qinlock), rituximab (Rituxan), rituximab and hyaluronidase human (Rituxan Hycela), romidepsin (Istodax), rucaparib camsylate (Rubraca), ruxolitinib phosphate (Jakafi), sacituzumab govitecan-hziy (Trodelvy), selinexor (Xpovio), selpercatinib (Retevmo), sclumetinib sulfate (Kosclugo), siltuximab (Sylvant), sirolimus protein-bound particles (Fyarro), sonidegib (Odomzo), sorafenib tosylate (Nexavar), sotorasib (Lumakras), sunitinib malate (Sutent), tafasitamab-cxix (Monjuvi), tagraxofusp-crzs (Elzonris), talazoparib tosylate (Talzenna), tamoxifen citrate (Soltamox), tazemetostat hydrobromide (Tazverik), tebentafusp-tebn (Kimmtrak), teclistamab-cqyv (Tecvayli), temsirolimus (Torisel), tepotinib hydrochloride (Tepmetko), tisagenlecleucel (Kymriah), tisotumab vedotin-tftv (Tivdak), tivozanib hydrochloride (Fotivda), toremifene (Fareston), trametinib (Mekinist), tramctinib dimethyl sulfoxide (Mekinist), trastuzumab (Herceptin), tremelimumab-actl (Imjudo), tretinoin (Vesanoid), tucatinib (Tukysa), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap).

In certain embodiments, the therapy administered to a subject comprises at least one chemotherapy drug. In some embodiments, the chemotherapy drug may comprise alkylating agents (for example, but not limited to, Chlorambucil, Cyclophosphamide, Cisplatin and Carboplatin), nitrosoureas (for example, but not limited to, Carmustine and Lomustine), anti-metabolites (for example, but not limited to, Fluorauracil, Methotrexate and Fludarabine), plant alkaloids and natural products (for example, but not limited to, Vincristine, Paclitaxel and Topotecan), anti-tumor antibiotics (for example, but not limited to, Bleomycin, Doxorubicin and Mitoxantrone), hormonal agents (for example, but not limited to, Prednisone, Dexamethasone, Tamoxifen and Leuprolide) and biological response modifiers (for example, but not limited to, Herceptin and Avastin, Erbitux and Rituxan). In some embodiments, the chemotherapy administered to a subject may comprise FOLFOX or FOLFIRI. In certain embodiments, a therapy may be administered to a subject that comprises at least one PARP inhibitor. In certain embodiments, the PARP inhibitor may include OLAPARIB, TALAZOPARIB, RUCAPARIB, NIRAPARIB (trade name ZEJULA), among others. Typically, therapies include at least one immunotherapy (or an immunotherapeutic agent). Immunotherapy refers generally to methods of enhancing an immune response against a given cancer type. In certain embodiments, immunotherapy refers to methods of enhancing a T cell response against a tumor or cancer.

In some embodiments, therapy is customized based on the status of a nucleic acid variant as being of somatic or germline origin. In some embodiments, essentially any cancer therapy (e.g., surgical therapy, radiation therapy, chemotherapy, immunotherapy, and/or the like) may be included as part of these methods. Customized therapies can include at least one immunotherapy (or an immunotherapeutic agent). Immunotherapy refers generally to methods of enhancing an immune response against a given cancer type. In certain embodiments, immunotherapy refers to methods of enhancing a T cell response against a tumor or cancer.

In some embodiments, the immunotherapy or immunotherapeutic agent targets an immune checkpoint molecule. Certain tumors are able to evade the immune system by co-opting an immune checkpoint pathway. Thus, targeting immune checkpoints has emerged as an effective approach for countering a tumor's ability to evade the immune system and activating anti-tumor immunity against certain cancers. Pardoll, Nature Reviews Cancer, 2012, 12:252-264.

In certain embodiments, the immune checkpoint molecule is an inhibitory molecule that reduces a signal involved in the T cell response to antigen. For example, CTLA4 is expressed on T cells and plays a role in downregulating T cell activation by binding to CD80 (aka B7.1) or CD86 (aka B7.2) on antigen presenting cells. PD-1 is another inhibitory checkpoint molecule that is expressed on T cells. PD-1 limits the activity of T cells in peripheral tissues during an inflammatory response. In addition, the ligand for PD-1 (PD-L1 or PD-L2) is commonly upregulated on the surface of many different tumors, resulting in the downregulation of anti-tumor immune responses in the tumor microenvironment. In certain embodiments, the inhibitory immune checkpoint molecule is CTLA4 or PD-1. In other embodiments, the inhibitory immune checkpoint molecule is a ligand for PD-1, such as PD-L1 or PD-L2. In other embodiments, the inhibitory immune checkpoint molecule is a ligand for CTLA4, such as CD80 or CD86. In other embodiments, the inhibitory immune checkpoint molecule is lymphocyte activation gene 3 (LAG3), killer cell immunoglobulin like receptor (KIR), T cell membrane protein 3 (TIM3), galectin 9 (GAL9), or adenosine A2a receptor (A2aR).

Antagonists that target these immune checkpoint molecules can be used to enhance antigen-specific T cell responses against certain cancers. Accordingly, in certain embodiments, the immunotherapy or immunotherapeutic agent is an antagonist of an inhibitory immune checkpoint molecule. In certain embodiments, the inhibitory immune checkpoint molecule is PD-1. In certain embodiments, the inhibitory immune checkpoint molecule is PD-L1. In certain embodiments, the antagonist of the inhibitory immune checkpoint molecule is an antibody (e.g., a monoclonal antibody). In certain embodiments, the antibody or monoclonal antibody is an anti-CTLA4, anti-PD-1, anti-PD-L1, or anti-PD-L2 antibody. In certain embodiments, the antibody is a monoclonal anti-PD-1 antibody. In some embodiments, the antibody is a monoclonal anti-PD-L1 antibody. In certain embodiments, the monoclonal antibody is a combination of an anti-CTLA4 antibody and an anti-PD-1 antibody, an anti-CTLA4 antibody and an anti-PD-L1 antibody, or an anti-PD-L1 antibody and an anti-PD-1 antibody. In certain embodiments, the anti-PD-1 antibody is one or more of pembrolizumab (Keytruda®) or nivolumab (Opdivo®). In certain embodiments, the anti-CTLA4 antibody is ipilimumab (Yervoy®). In certain embodiments, the anti-PD-L1 antibody is one or more of atezolizumab (Tecentriq®), avelumab (Bavencio®), or durvalumab (Imfinzi®).

In certain embodiments, the immunotherapy or immunotherapeutic agent is an antagonist (e.g., antibody) against CD80, CD86, LAG3, KIR, TIM3, GAL9, or A2aR. In other embodiments, the antagonist is a soluble version of the inhibitory immune checkpoint molecule, such as a soluble fusion protein comprising the extracellular domain of the inhibitory immune checkpoint molecule and an Fc domain of an antibody. In certain embodiments, the soluble fusion protein comprises the extracellular domain of CTLA4, PD-1, PD-L1, or PD-L2. In some embodiments, the soluble fusion protein comprises the extracellular domain of CD80, CD86, LAG3, KIR, TIM3, GAL9, or A2aR. In one embodiment, the soluble fusion protein comprises the extracellular domain of PD-L2 or LAG3.

In certain embodiments, the immune checkpoint molecule is a co-stimulatory molecule that amplifies a signal involved in a T cell response to an antigen. For example, CD28 is a co-stimulatory receptor expressed on T cells. When a T cell binds to antigen through its T cell receptor, CD28 binds to CD80 (aka B7.1) or CD86 (aka B7.2) on antigen-presenting cells to amplify T cell receptor signaling and promote T cell activation. Because CD28 binds to the same ligands (CD80 and CD86) as CTLA4, CTLA4 is able to counteract or regulate the co-stimulatory signaling mediated by CD28. In certain embodiments, the immune checkpoint molecule is a co-stimulatory molecule selected from CD28, inducible T cell co-stimulator (ICOS), CD137, OX40, or CD27. In other embodiments, the immune checkpoint molecule is a ligand of a co-stimulatory molecule, including, for example, CD80, CD86, B7RP1, B7-H3, B7-H4, CD137L, OX40L, or CD70.

Agonists that target these co-stimulatory checkpoint molecules can be used to enhance antigen-specific T cell responses against certain cancers. Accordingly, in certain embodiments, the immunotherapy or immunotherapeutic agent is an agonist of a co-stimulatory checkpoint molecule. In certain embodiments, the agonist of the co-stimulatory checkpoint molecule is an agonist antibody and preferably is a monoclonal antibody. In certain embodiments, the agonist antibody or monoclonal antibody is an anti-CD28 antibody. In other embodiments, the agonist antibody or monoclonal antibody is an anti-ICOS, anti-CD137, anti-OX40, or anti-CD27 antibody. In other embodiments, the agonist antibody or monoclonal antibody is an anti-CD80, anti-CD86, anti-B7RP1, anti-B7-H3, anti-B7-H4, anti-CD137L, anti-OX40L, or anti-CD70 antibody.

In certain embodiments, the status of a nucleic acid variant from a sample from a subject as being of somatic or germline origin may be compared with a database of comparator results from a reference population to identify customized or targeted therapies for that subject. Typically, the reference population includes patients with the same cancer or disease type as the subject and/or patients who are receiving, or who have received, the same therapy as the subject. A customized or targeted therapy (or therapies) may be identified when the nucleic variant and the comparator results satisfy certain classification criteria (e.g., are a substantial or an approximate match).

In certain embodiments, the customized therapies described herein are typically administered parenterally (e.g., intravenously or subcutaneously). Pharmaceutical compositions containing an immunotherapeutic agent are typically administered intravenously. Certain therapeutic agents are administered orally. However, customized therapies (e.g., immunotherapeutic agents, etc.) may also be administered by any method known in the art, for example, buccal, sublingual, rectal, vaginal, intraurethral, topical, intraocular, intranasal, and/or intraauricular, which administration may include tablets, capsules, granules, aqueous suspensions, gels, sprays, suppositories, salves, ointments, or the like.

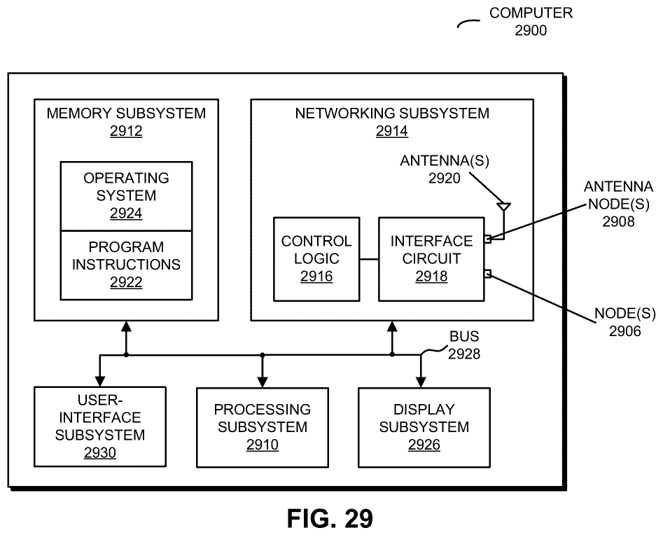

We now describe embodiments of a computer in a computer system, which may perform at least some of the operations or instructions for at least some of the operations in the analysis techniques. FIG. 29 presents a block diagram illustrating an example of a computer 2900, e.g., in a computer system (such as computer system 100 in FIG. 1), in accordance with some embodiments. Computer 2900 may regulate various aspects sample preparation, sequencing, and/or analysis. In some examples, computer 2900 is configured to perform sample preparation and sample analysis, including nucleic acid sequencing.

Computer 2900 may include: one of computers 110. This computer may include processing subsystem 2910, memory subsystem 2912, and networking subsystem 2914. Processing subsystem 2910 includes one or more devices configured to perform computational operations. For example, processing subsystem 2910 can include one or more microprocessors (such as a single-core or a multi-core processor), ASICs, microcontrollers, programmable-logic devices, GPUs and/or one or more DSPs. Processing subsystem 2910 may perform parallel processing of one or more operations in the analysis techniques. Note that a given component in processing subsystem 2910 are sometimes referred to as a ‘computation device’.

Memory subsystem 2912 includes one or more devices for storing data and/or instructions for processing subsystem 2910 and networking subsystem 2914. For example, memory subsystem 2912 can include dynamic random access memory (DRAM), static random access memory (SRAM), flash and/or other types of memory. In some embodiments, instructions for processing subsystem 2910 in memory subsystem 2912 include: program instructions or sets of instructions (such as program instructions 2922 or operating system 2924), which may be executed by processing subsystem 2910. Note that the one or more computer programs or program instructions may constitute a computer-program mechanism. Moreover, instructions in the various program instructions in memory subsystem 2912 may be implemented in: a high-level procedural language, an object-oriented programming language, and/or in an assembly or machine language. Furthermore, the programming language may be compiled or interpreted, e.g., configurable or configured (which may be used interchangeably in this discussion), to be executed by processing subsystem 2910. Thus, program instructions 2922 may be precompiled for use with computer 2900 or may be compiled at runtime. In some embodiments, program instructions 2922 are stored or embodied on a type of non-transitory machine-readable medium, which may include a portable non-transitory machine-readable medium (e.g., a floppy disk, a flexible disk, hard disk, magnetic tape, any other magnetic medium, a CD-ROM, DVD or DVD-ROM, any other optical medium, punch cards, paper tape, any other physical storage medium with patterns of holes, a RAM, a ROM, a PROM and EPROM, a FLASH-EPROM, any other memory chip or cartridge, or any other medium from which a computer may read programming code and/or data).

In addition, memory subsystem 2912 can include mechanisms for controlling access to the memory. In some embodiments, memory subsystem 2912 includes a memory hierarchy that includes one or more caches coupled to a memory in computer 2900. In some of these embodiments, one or more of the caches is located in processing subsystem 2910.

In some embodiments, memory subsystem 2912 is coupled to one or more high-capacity mass-storage devices (not shown), which may be external to computer 2900 and/or remotely located (and, thus, accessed via a network). For example, memory subsystem 2912 can be coupled to a magnetic or optical drive, a solid-state drive, or another type of mass-storage device. In these embodiments, memory subsystem 2912 can be used by computer 2900 as fast-access storage for often-used data, while the mass-storage device is used to store less frequently used data. Note that data may be transferred from one location to another using. e.g., a network (such as the Internet and/or an intra-net) or physical data transfer (e.g., using a hard drive, thumb drive, or other data-storage device).

Networking subsystem 2914 includes one or more devices configured to couple to and communicate on a wired and/or wireless network (i.e., to perform network operations), including: control logic 2916, an interface circuit 2918 and one or more antennas 2920 (or antenna elements). (While FIG. 29 includes one or more antennas 2920, in some embodiments computer 2900 includes one or more nodes, such as antenna nodes 2908, e.g., a metal pad or a connector, which can be coupled to the one or more antennas 2920, or nodes 2906, which can be coupled to a wired or optical connection or link. Thus, computer 2900 may or may not include the one or more antennas 2920. Note that the one or more nodes 2906 and/or antenna nodes 2908 may constitute input(s) to and/or output(s) from computer 2900.) For example, networking subsystem 2914 can include a Bluetooth™ networking system, a cellular networking system (e.g., a 3G/4G/5G network such as UMTS, LTE, etc.), a universal serial bus (USB) networking system, a networking system based on the standards described in IEEE 802.11 (e.g., a Wi-Fi® networking system), an Ethernet networking system, and/or another networking system.

Networking subsystem 2914 includes processors, controllers, radios/antennas, sockets/plugs, and/or other devices used for coupling to, communicating on, and handling data and events for each supported networking system. Note that mechanisms used for coupling to, communicating on, and handling data and events on the network for each network system are sometimes collectively referred to as a ‘network interface’ for the network system. Moreover, in some embodiments a ‘network’ or a ‘connection’ between the electronic devices does not yet exist. Therefore, computer 2900 may use the mechanisms in networking subsystem 2914 for performing simple wireless communication between electronic devices, e.g., transmitting advertising or beacon frames and/or scanning for advertising frames transmitted by other electronic devices.

Within computer 2900, processing subsystem 2910, memory subsystem 2912, and networking subsystem 2914 are coupled together using bus 2928. Bus 2928 may include an electrical, optical, and/or electro-optical connection that the subsystems can use to communicate commands and data among one another. Although only one bus 2928 is shown for clarity, different embodiments can include a different number or configuration of electrical, optical, and/or electro-optical connections among the subsystems.

In some embodiments, computer 2900 includes a display subsystem 2926 for displaying information on a display, which may include a display driver and the display, such as a liquid-crystal display, a multi-touch touchscreen, etc. Moreover, computer 2900 may include a user-interface subsystem 2930, such as: a mouse, a keyboard, a trackpad, a stylus, a voice-recognition interface, and/or another human-machine interface. Note that user-interface subsystem 2930 may include graphical user interface (GUI) and/or a web-based user interface

Additional details relating to computer systems and networks, data structures, databases, and computer program products are also provided in, for example, Peterson, Computer Networks: A Systems Approach, Morgan Kaufmann, 5th Ed. (2011), Kurose, Computer Networking: A Top-Down Approach, Pearson, 7^{th} Ed. (2016), Elmasri, Fundamentals of Database Systems, Addison Wesley, 6th Ed. (2010), Coronel, Database Systems: Design. Implementation, & Management, Cengage Learning, 11^{th} Ed. (2014), Tucker, Programming Languages, McGraw-Hill Science/Engineering/Math, 2nd Ed. (2006), and Rhoton, Cloud Computing Architected: Solution Design Handbook. Recursive Press (2011), each of which is hereby incorporated by reference in its entirety.

Computer 2900 can be (or can be included in) any electronic device with at least one network interface. For example, computer 2900 can be (or can be included in): a desktop computer, a laptop computer, a subnotebook/netbook, a server, a supercomputer, a tablet computer, a smartphone, a cellular telephone, a consumer-electronic device, a portable computing device, communication equipment, and/or another electronic device.

Although specific components are used to describe computer 2900, in alternative embodiments, different components and/or subsystems may be present in computer 2900. For example, computer 2900 may include one or more additional processing subsystems, memory subsystems, networking subsystems, and/or display subsystems. Additionally, one or more of the subsystems may not be present in computer 2900. Moreover, in some embodiments, computer 2900 may include one or more additional subsystems that are not shown in FIG. 29. Also, although separate subsystems are shown in FIG. 29, in some embodiments some or all of a given subsystem or component can be integrated into one or more of the other subsystems or component(s) in computer 2900. For example, in some embodiments program instructions 2922 are included in operating system 2924 and/or control logic 2916 is included in interface circuit 2918.

Moreover, the circuits and components in computer 2900 may be implemented using any combination of analog and/or digital circuitry, including: bipolar, PMOS and/or NMOS gates or transistors. Furthermore, signals in these embodiments may include digital signals that have approximately discrete values and/or analog signals that have continuous values. Additionally, components and circuits may be single-ended or differential, and power supplies may be unipolar or bipolar.

An integrated circuit may implement some or all of the functionality of networking subsystem 2914 and/or computer 2900. The integrated circuit may include hardware and/or software mechanisms that are used for transmitting signals from computer 2900 and receiving signals at computer 2900 from other electronic devices. Aside from the mechanisms herein described, radios are generally known in the art and hence are not described in detail. In general, networking subsystem 2914 and/or the integrated circuit may include one or more radios.

In some embodiments, an output of a process for designing the integrated circuit, or a portion of the integrated circuit, which includes one or more of the circuits described herein may be a computer-readable medium such as, e.g., a magnetic tape or an optical or magnetic disk or solid state disk. The computer-readable medium may be encoded with data structures or other information describing circuitry that may be physically instantiated as the integrated circuit or the portion of the integrated circuit. Although various formats may be used for such encoding, these data structures are commonly written in: Caltech Intermediate Format (CIF), Calma GDS II Stream Format (GDSII), Electronic Design Interchange Format (EDIF), OpenAccess (OA), or Open Artwork System Interchange Standard (OASIS). Those of skill in the art of integrated circuit design can develop such data structures from schematics of the type detailed above and the corresponding descriptions and encode the data structures on the computer-readable medium. Those of skill in the art of integrated circuit fabrication can use such encoded data to fabricate integrated circuits that include one or more of the circuits described herein.

While some of the operations in the preceding embodiments were implemented in hardware or software, in general the operations in the preceding embodiments can be implemented in a wide variety of configurations and architectures. Therefore, some or all of the operations in the preceding embodiments may be performed in hardware, in software or both. For example, at least some of the operations in the analysis techniques may be implemented using program instructions 2922, operating system 2924 (such as a driver for interface circuit 2918) or in firmware in interface circuit 2918. Thus, the analysis techniques may be implemented at runtime of program instructions 2922. Alternatively or additionally, at least some of the operations in the analysis techniques may be implemented in a physical layer, such as hardware in interface circuit 2918.

Note that the use of the phrases ‘capable of,’ ‘capable to,’ ‘operable to,’ or ‘configured to’ in one or more embodiments, refers to some apparatus, logic, hardware, and/or element designed in such a way to enable use of the apparatus, logic, hardware, and/or element in a specified manner.

In the preceding description, we refer to ‘some embodiments’. Note that ‘some embodiments’ describes a subset of all of the possible embodiments, but does not always specify the same subset of embodiments. Moreover, note that the numerical values provided are intended as illustrations of the analysis techniques. In other embodiments, the numerical values can be modified or changed.

The foregoing description is intended to enable any person skilled in the art to make and use the disclosure, and is provided in the context of a particular application and its requirements. Moreover, the foregoing descriptions of embodiments of the present disclosure have been presented for purposes of illustration and description only. They are not intended to be exhaustive or to limit the present disclosure to the forms disclosed. Accordingly, many modifications and variations will be apparent to practitioners skilled in the art, and the general principles defined herein may be applied to other embodiments and applications without departing from the spirit and scope of the present disclosure. Additionally, the discussion of the preceding embodiments is not intended to limit the present disclosure. Thus, the present disclosure is not intended to be limited to the embodiments shown, but is to be accorded the widest scope consistent with the principles and features disclosed herein.

What is claimed is:

1. A method for processing a sample, comprising:
receiving or collecting molecules of deoxyribonucleic acid (DNA) in the sample;
attaching adapters to ends of the molecules of DNA;
selectively cutting, in one or more off-target regions of the molecules of DNA, wherein a given cut results in fragment ends in the molecules of DNA;
depleting or removing the off-target regions in the molecules of DNA, wherein the off-target regions in the molecules of DNA are different from target regions in the molecules of DNA having methylation or binding states;
attaching, to a remainder of the molecules of DNA following the depleting or removing operation, motor adapters to locations on the remainder of the molecules of DNA; and
analyzing the one or more target regions by pulling or extracting the remainder of the molecules of DNA through an analysis membrane using the motor adapters.

2. The method of claim 1, wherein the adapters prevent the selective cutting.

3. The method of claim 2, wherein the adapters comprise ligation adapters; and
wherein a given off-target region is separated from a given target region by a given ligation adapter.

4. The method of claim 1, wherein the selective cutting uses a Cas9 protein or a restriction enzyme.

5. The method of claim 1, wherein the depletion or removal comprises:
digesting the off-target regions in the molecules of DNA; or
pulling or extracting out of solution the digested off-target regions in the molecules of DNA.

6. The method of claim 5, wherein the digestion comprises exonuclease digestion.

7. The method of claim 5, wherein the pulling or extracting out uses magnetic beads.

8. The method of claim 7, wherein the magnetic beads comprise streptavidin magnetic beads.

9. The method of claim 1, wherein the off-target regions comprise at least a subset of the fragment ends in the molecules of DNA.

10. The method of claim 1, wherein the analysis membrane comprises a nanopore membrane.

11. The method of claim 1, wherein the molecules of DNA comprise cell-free DNA having lengths less than 200 bases.

12. The method of claim 1, wherein the method excludes amplification of the one or more target regions.

13. The method of claim 1, wherein the analysis comprises determining the molecules of DNA in the one or more target regions.

14. The method of claim 13, wherein the analysis comprises determining the methylation states or the binding states of the one or more target regions.

15. A computer system, comprising:
an interface circuit;
a computation device coupled to the interface circuit; and
memory, coupled to the computation device, configured to store program instructions, wherein, when executed by the computation device, the program instructions cause the computer system to perform one or more operations comprising:
receiving or collecting molecules of deoxyribonucleic acid (DNA) in the sample;
providing an instruction to attach adapters to ends of the molecules of DNA;
providing a second instruction to selectively cut, in one or more off-target regions, the molecules of DNA, wherein a given cut results in fragment ends in the molecules of DNA;
providing a third instruction to deplete or remove the off-target regions in the molecules of DNA, wherein the off-target regions in the molecules of DNA are different from target regions in the molecules of DNA having methylation or binding states;
providing a fourth instruction to attach, to a remainder of the molecules of DNA following the depleting or removing operation, motor adapters to locations on the remainder of the molecules of DNA; and
analyzing the one or more target regions by pulling or extracting the remainder of the molecules of DNA through an analysis membrane using the motor adapters.

16. The computer system of claim 15, wherein the adapters prevent the selective cutting.

17. The computer system of claim 15, wherein the selective cutting uses a Cas9 protein or a restriction enzyme.

18. The computer system of claim 15, wherein the off-target regions comprise at least a subset of the fragment ends in the molecules of DNA.

19. A non-transitory computer-readable storage medium for use in conjunction with a computer system, the computer-readable storage medium configured to store program instructions that, when executed by the computer system, causes the computer system to perform one or more operations comprising:
receiving or collecting molecules of deoxyribonucleic acid (DNA) in the sample;
providing an instruction to attach adapters to ends of the molecules of DNA;
providing a second instruction to selectively cut, in one or more off-target regions, the molecules of DNA, wherein a given cut results in fragment ends in the molecules of DNA;
providing a third instruction to remove or deplete or remove the off-target regions in the molecules of DNA, wherein the off-target regions in the molecules of DNA are different from target regions in the molecules of DNA having methylation or binding states;
providing a fourth instruction to attach, to a remainder of the molecules of DNA following the depleting or removing operation, motor adapters to locations on the remainder of the molecules of DNA; and
analyzing the one or more target regions by pulling or extracting the remainder of the molecules of DNA through an analysis membrane using the motor adapters.

20. The non-transitory computer-readable storage medium of claim 19, wherein the adapters prevent the selective cutting.
